SEQUENCE LISTING (4 sequences)

SEQ ID NO: 1 - DNA, 44 bp, synthetic construct
  source 1..44: mol_type = other DNA
tactcagggcactgcaagcaattgtggtcccaatgggctgagta

SEQ ID NO: 2 - DNA, 28 bp, synthetic construct
  source 1..28: mol_type = other DNA
agtccgtggtagggcaggttggggtgac

SEQ ID NO: 3 - DNA, 49 bp, synthetic construct
  source 1..49: mol_type = other DNA
  modified_base 6: mod_base = OTHER; note = Black hole quencher 2 modified nucleotide
ttttttactcagggcactgcaagcaattgtggtcccaatgggctgagta

SEQ ID NO: 4 - DNA, 35 bp, synthetic construct
  source 1..35: mol_type = other DNA
  modified_base 6: mod_base = OTHER; note = Black hole quencher 1 modified nucleotide
ttttttagtccgtggtagggcaggttggggtgact

(19) United States
(12) Patent Application Publication
(10) Pub. No.: US 2025/0314646 A1
(43) Pub. Date: Oct. 9, 2025

(54) SYSTEMS AND METHODS FOR ANALYTE DETECTION IN BIOLOGICAL SOLUTIONS

(71) Applicant: eGlint, Inc., La Jolla, CA (US)

(72) Inventors: Miena ARMANIOUS, La Jolla, CA (US); Denis MALYSHEV, La Jolla, CA (US); Floyd ROMESBERG, La Jolla, CA (US)

(73) Assignee: eGlint, Inc., La Jolla, CA (US)

(21) Appl. No.: 18/844,182

(22) PCT Filed: Mar. 9, 2023
(86) PCT No.: PCT/US2023/064017

§ 371 Date: Sep. 5, 2024

Related U.S. Application Data
(60) Provisional application No. 63/318,658, filed on Mar. 10, 2022.

(51) Int. Cl. G01N 33/533; G01N 15/1434; G01N 33/543

(52) U.S. Cl. CPC G01N 33/533; G01N 15/1436; G01N 33/54366

(57) ABSTRACT

The inventions relate to biosensors, in particular biosensors for detecting the presence of an analyte in a sample using a surface-immobilized molecule. The principle of detecting the analyte is based on measuring the persistence of the state (or ensemble of states) of the surface-immobilized molecule with, and without the presence of a binding partner, upon the change in environment about the surface-immobilized molecule.

### CROSS REFERENCE TO RELATED APPLICATIONS

This document claims priority to U.S. Prov. Ser. No. 63/318,658, filed Mar. 10, 2022, the contents of which are hereby incorporated by reference in their entirety.

### SEQUENCE LISTING

The instant application contains a Sequence Listing which has been submitted electronically in XML format and is hereby incorporated by reference in its entirety. Said XML copy, created on May 8, 2025, is named EGL_002US_SL.xml and is 5,757 bytes in size.

### FIELD

The inventions relate to biosensors. In particular, this disclosure relates to systems and methods for measuring the change of persistence of binding competent states of surface-immobilized biomolecules as a method for the detection of analytes.

### TECHNICAL BACKGROUND

The following includes information that may be useful in understanding the present invention. It is not an admission that any of the information, publications, or documents specifically or implicitly referenced herein is prior art, or essential, to the presently described or claimed inventions. All publications, patents, related applications, and other written or electronic materials mentioned or identified herein are hereby incorporated herein by reference in their entirety. The information incorporated is as much a part of the application as filed as if all of the text and other content was repeated in the application, and should be treated as part of the text and content of the application as filed.

Developing biosensors to detect an analyte requires that general methods be available to generate sensor molecules that recognize the desired analyte with high specificity. This challenge has been met in recent years with the development of biological sensors (“biosensors”), including proteins (e.g. antibodies or lectins), nucleic acids (aptamers), or carbohydrates. However, detecting the analyte also requires that analyte binding to the sensor molecules induces a biophysical change in the sensor molecules such that it generates a detectable signal. While many methods have been explored, they are commonly challenged by unpredictable 2 or insufficiently large or specific changes in the relevant biophysical property of the sensor molecule.

In light of the above, there remains a need for accurate and rapid measurement of analytes.

### SUMMARY OF THE INVENTION

The inventions described and claimed herein have many attributes and embodiments including, but not limited to, those set forth or described or referenced in this Brief Summary. It is not intended to be all-inclusive, and the inventions described and claimed herein are not limited to or by the features or embodiments identified in this introduction, which are included for purposes of illustration only and not necessarily restriction.

It is an object of the invention to provide systems and methods for measuring the persistence or change of persistence of binding competent states of immobilized molecules when in the presence or absence of their respective binding partners as a means to detect the binding partners (e.g., analytes).

Also featured in one aspect of this invention are methods of detecting the presence and concentration of a binding partner in a sample suspected of or having said binding partner, the method comprising: (a) contacting an immobilized molecule that has a binding competent state that binds to a binding partner with a sample suspected of or having said binding partner such that when the binding partner is present, the binding partner forms a complex with the immobilized molecule; (b) measuring the persistence or change of persistence of the binding competent state of the immobilized molecule in response to a change in its environment; (c) comparing the persistence of the binding competent state to that in the absence of said binding partner, where when the persistence of the binding competent state is about the same as that in the absence of the binding partner, the binding partner is absent; and when the persistence of the binding competent state is different than that in the absence of the binding partner, the change of persistence indicates that the binding partner is present. In some aspects, detecting the concentration of an analyte can be a quantitative measurement.

In some aspects, the persistence can be measured after the immobilized molecule forms the complex with the binding partner. In some aspects, the persistence can be measured before the immobilized molecule forms the complex with the binding partner.

In some aspects, the persistence can be measured continuously while the immobilized molecule is responding to the change in its environment.

In some aspects, the persistence can be measured by measuring one or a plurality of physical characteristics of the immobilized molecule and the binding partner. One of the physical characteristics can be the mass of the immobilized molecule, and can be measured using surface acoustic wave measurements, surface plasmon resonance (SPR), or bilayer interferometry (BLI). One of the physical characteristics can be the structure of the immobilized molecule, and can be measured using AFM (atomic force microscopy) or STM (scanning tunneling microscopy). In some aspects, the change in the environment can be applied in a stepwise or gradual manner.

In some aspects, in step (b), the environment change can include or exclude a change of: the concentration of chemical agents which can include or exclude negative and positive ions, detergents, chaotropic agents; pH, electric field, temperature, magnetic field, ionic strength, light intensity, light polarization, light wavelength, or shear force.

In some aspects, the binding partner can be an ion, small molecule, peptide, protein, synthetic polymer, antibody, cell, virus, organelle, nucleic acid, oligosaccharide, glycoprotein, or component thereof. In some aspects, when the binding partner is a nucleic acid, the immobilized molecule is not a nucleic acid. In some aspects, when the immobilized molecule is a nucleic acid, the binding partner is not a nucleic acid. In some aspects, the immobilized molecule in the binding competent state can bind to the binding partner with a dissociation constant (K_{d}) of less than 10\hat{ }-6 M (1 micromolar). In some aspects, the immobilized molecule can be a protein (e.g. antibody or lectin), carbohydrate, small molecule, synthetic polymer, peptide, or nucleic acid. In some aspects, the nucleic acid can be an aptamer, in particular an RNA aptamer, DNA aptamer, or modified nucleic acid aptamer.

In some aspects, the immobilized molecule has a tertiary structure. In some aspects, the persistence can be measured by detecting a conformational change in the immobilized molecule. In some aspects, the persistence can be measured by characterizing the structure of the immobilized molecule. In some aspects, the immobilized molecule can comprise a dye or dye pair conjugated to the immobilized molecule. In some aspects, the dye pair can be a donor-acceptor fluorophore pair, in particular a FRET pair (Forster resonance energy transfer). In some aspects, the dye pair can be a fluorophore-quencher pair. In some aspects, each of the dyes can be conjugated to a separate site on the immobilized molecule.

In some aspects, the immobilized molecule can be a nucleic acid sequence comprising one or a plurality of dyes, wherein the dyes are conjugated to a modified nucleotide within the nucleic acid sequence. In some aspects, the immobilized molecule can be an aptamer. In some aspects, the aptamer can further comprise a functional group which can form a conjugation site to a small molecule or a surface (which can include or exclude a dye). In some aspects, the immobilized molecule can be a protein, and the dyes are conjugated to the protein by chemical linkage to one or a plurality of canonical or non-canonical proteogenic amino acids within the protein. In some aspects, at least one of the non-canonical proteogenic amino acids can comprise a biorthogonal reactive moiety. In some aspects, at least one of the non-canonical proteogenic amino acids can provide a site for conjugation. The persistence can be measured by measuring a photo-physical property (e.g., fluorescence intensity, wavelength, polarization, photoluminescence lifetime, chemiluminescence, or anisotropy) of one or more of the dyes of the dye pair. The photophysical property can be measured using an electronic sensor. In some aspects, the electronic sensor is a transducer that converts photons to electrons. In some aspects, the electronic sensor can comprise CMOS (complementary metal-oxide semiconductor) photodiodes or CCD (charged coupled detector) photodiodes.

In some aspects, the sample can be from a bodily fluid. The bodily fluid can be from nasal turbinate, ocular fluid, cerebral spinal fluid, urine, feces, diarrhea, bone marrow, blood, plasma, saliva, homogenized tissue, or sweat.

In some aspects, the binding competent state of the immobilized molecule can have a lower free energy state when the immobilized molecule is in a complex with the binding partner than when not in a complex with the binding partner.

In some aspects, the step of comparing the persistence of the binding competent state to that in the absence of said binding partner to assess a change in persistence can be performed using a computer processor.

Also featured in one aspect of this invention is a method of detecting the presence of an analyte in a sample suspected of or having said analyte, the method comprising: (a) applying an excitation light and an unfolding force such as electric field or an increase in temperature to an immobilized aptamer which comprises a dye pair and has one or a plurality of binding competent state(s); (b) measuring the persistence of one or a plurality of binding competent state(s) of the aptamer in the presence of the excitation light and applied unfolding force; (c) removing the applied unfolding force; (d) contacting the immobilized aptamer with a sample suspected of or having said analyte such that when the analyte is present, the analyte forms a complex with the immobilized aptamer; (e) measuring the persistence of the binding competent state(s) of the immobilized aptamer in the presence of the sample upon application of the unfolding force; (f) comparing the persistence of the binding competent state(s) of the immobilized aptamer in the presence and the absence of the sample, when the persistence of the binding competent state(s) is about the same as that in the absence of the sample, the analyte is absent; and when the persistence of the binding competent state(s) is different than that in the absence of the sample, the analyte is present. In some aspects, the persistence of the binding competent state(s) can be greater in the presence of the sample than that in the absence of the sample.

Also featured in one aspect of this invention is a biosensor comprising: (a) an excitation light source; (b) a detection device comprising a plurality of sets of stacked layers, comprising: (i) a first set of stacked layers comprising a top layer which is a transparent layer configured to support an immobilized aptamer (which comprises a dye pair and has a binding competent state(s)); and (ii) a second set of stacked layers comprising an optical filter and a solid-state photodetector array; wherein the optical filter is operably coupled to the transparent conductive layer and the solid-state photodetector array, wherein the immobilized aptamer comprises a nucleic acid sequence, a dye pair, and one or a plurality of binding competent state(s), wherein the optical filter comprises a plurality of opaque walls which define a field of view for the photodetectors of the solid-state photodetector array, wherein the optical filter comprises a plurality of a set of filter layers in between the opaque walls which define a transmission spectral band of the light traveling within the defined field of view, wherein the dye pair is configured to be positioned within the field of view of the solid-state photodetector array and the excitation light source is configured to be exterior to the field of view of the solid-state photodetector array, and wherein the filter layers are configured to transmit the emitted light from the dye pair to the solid-state photodetector array when the dye pair is subjected to an excitation light, and to block light outside the emission spectral band of the dye pair. The operable coupling between the optical filter and the transparent layer can be a physical connection, a photonic connection, and/or an electrical connection. In some aspects, the transparent layer can be electrically conductive. In some aspects, the dye pair of the biosensor can be covalently linked to the nucleic acid sequence. In some aspects, the walls which define a field of view can be essentially optically opaque. In some aspects, the solid-state photodiode array can be a CMOS imaging sensor. In some aspects, the solid-state photodiode array can be configured to detect the emission signal from the dye pair when the dye pair is subject to light from the excitation light source.

Also featured is a biosensor comprising an excitation light source; an imaging device that is configured to convert an optical signal into an electric signal; a conductive layer comprising one or a plurality of clusters of immobilized molecules each of which comprises a dye or dye pair in a medium; an environmental perturbation apparatus that is configured to perturb the environment of the immobilized molecules; and an optical coupling medium configured to be positioned between the one or plurality of clusters of immobilized molecules and the imaging device. In some aspects, the excitation source optionally comprises an optical filter which defines an illumination wavelength and bandwidth. In some aspects, the optical coupling medium is configured to transmit the optical signal from the immobilized molecule and block the background optical signal from the excitation light source. In some aspects, the optical coupling medium optionally comprises one or a plurality of an optical element selected from: spectral filters, diffraction gratings, light reflectors, light absorbers, and total internal reflection (TIR) structures, and wherein said optical element is configured to detect an optical signal from the immobilized molecules in the presence of background illumination from the excitation light source. In some aspects, the optical coupling medium optionally is physically connected to the imaging device and/or the conductive layer. In some aspects, the optical coupling medium optionally comprises an optical lens or is lens-less. In some aspects, the conductive layer is configured to transfer charges from the environmental perturbation apparatus to perturb the environment of the one or a plurality of clusters of immobilized molecules. In some aspects, the conductive layer optionally is configured to allow light coupling from the dye or dye pair to the optical coupling medium. In some aspects, the environmental perturbation apparatus optionally comprises a voltage sweep source which applies a time-varying voltage signal across the medium in which the one or a plurality of clusters of immobilized molecules is present. In some aspects, the imaging device is configured to convert a received optical signal into an electrical signal, wherein the strength of the electrical signal is directly dependent on the strength of the received optical signal after pathing through the optical coupling medium. In some aspects, the imaging device is a CMOS active-pixel sensor, CMOS passive-pixel sensor, CCD, or pinned-photodiode array.

In some aspects, the conductive layer is optically transparent. In some aspects, the operable coupling between the optical filter and the transparent conductive layer is a physical connection. In some aspects, the dye pair is covalently linked to the nucleic acid sequence. In some aspects, the field of view which is defined by the walls is essentially optically transparent to the wavelength(s) emitted by the emission light source.

In some aspects, the solid-state photodetector array is configured to detect the emission signal from the dye pair when the dye pair is subject to a light source from the excitation light source. In some aspects, the excitation source is configured to illuminate the cluster of immobilized molecules. In some aspects, the excitation source is configured to selectively illuminate the cluster of immobilized molecules.

In some aspects, the biosensor further comprising a data communication channel which is configured to transfer the electric signal data versus the voltage sweep profile to a computer processing unit which is programmed to compare the electrical signal originating from the bound and unbound aptamer clusters designed for a given target molecule.

Also featured in one aspect of this invention is an array comprising a plurality of biosensors as described herein. Also featured in one aspect of this invention is a kit comprising a biosensor as described herein.

Also disclosed herein are methods of assaying for an analyte in a sample. Some such methods comprise one or more of the steps of: contacting the sample to an affinity reagent having a first configuration, wherein the affinity reagent first configuration is sensitive to presence of the analyte; and assaying the configuration of the affinity regent.

The affinity reagent variously comprises an oligonucleotide, an aptamer, a protein, an antibody, or other target binding moiety. The aptamer comprises DNA or RNA in various embodiments. The aptamer is in some cases tethered to a surface, such as a planar surface or the surface of a bead. Alternately, in some cases the aptamer is in solution, such as in a well or an emulsion droplet.

The affinity reagent comprises a first binding moiety that binds the analyte at a first region, and in some cases comprises a second binding moiety that binds that analyte at a second region. In the case of aptamers, the first binding moiety and the second binding moiety in some cases share a common phosphodiester bond.

The sample is in some cases in solution. The sample is in some cases an aqueous sample, and may be a raw sample or may be buffered.

The assaying variously comprises disrupting the affinity reagent binding to its target molecule, for example by heating the affinity reagent, subjecting the affinity reagent to an electric field, subjecting the affinity reagent to a magnetic field, subjecting the affinity reagent to sonication, or subjecting the affinity reagent to acoustic waves.

The assaying comprises measuring the affinity reagent confirmation, such as by measuring fluorescence from the affinity reagent, or by measuring an electrochemical signal, such as is generated by redox ions.

In various embodiments, assaying comprises changing a condition and measuring an output of affinity reagent conformation such as fluorescence during the changing of the condition, or subsequent to the changing of the condition, or both during and subsequent to the changing of the condition.

Affinity reagents often comprise an aptamer, such as an aptamer that comprises a fluorophore, and in some cases further comprises a quencher, or a fluorophore acceptor pair.

For any of the embodiments discussed herein, in some cases a change in aptamer configuration comprises binding to the analyte, such as a change which stabilizes the aptamer configuration. Often, a change in aptamer configuration indicates presence of the analyte in the sample. The change in aptamer configuration results in an increased aptamer stability, and May result in an increase in fluorescence or other signal in response to a stability challenging treatment such as heat, electric field or other examples above or elsewhere herein, or alternately may result in an increased stability but a decreased fluorescence or other signal in response to said treatment.

For example, the change in aptamer configuration results in some cases in a change in a threshold at which the changing of the condition impacts aptamer fluorescence.

The assaying is completed in no more than 5 minutes, or no more than 5, 6, 7, 8, 9, 10, 15, 20, or 30 minutes, in particular when the change in the destabilizing condition is effected gradually or incrementally, such as through subjecting the sample to a gradient. Other durations are also consistent with the disclosure herein.

Alternately, the assaying is completed in no more than 30 seconds, such as when the assaying comprises assaying at a single destabilizing condition or changing from a first to a second and optionally to a third condition parameter. Exemplary times are no more than 10, 15, 20 30 45 or 60 seconds, or no more than 1, 2, 3, 4, or 5 minutes. Other durations are also consistent with the disclosure herein.

Assaying exhibits very high sensitivity in some embodiments, such that in some cases assaying is sensitive to an analyte at a concentration of at least 1 fM, or alternately at least 10 fM, 100 fM, 1 pM, 10 pM, 100 pM, 1 nM, 10 nM, 100 nM, 1 uM or greater than 1 uM.

A number of sample types are consistent with the disclosure herein, such as a body fluid such as blood, for example in a droplet of at least 1 μL, 2 μL, 5 μL, 10 μL, 20 μL, 50 μL, or greater than 50 μL. Alternate body fluids, such as plasma, saliva, sweat, bile, urine or other fluids are similarly consistent with the disclosure herein, such as in the volumes listed.

Disclosed herein are surfaces for detection of a target analyte, such as surfaces comprising one or more of the following elements: a plurality of aptamer clusters, wherein a first cluster of the plurality of clusters comprises a first aptamer having a first configuration, wherein the aptamer first configuration is sensitive to presence of a first analyte, and wherein a second cluster of the plurality of clusters comprises a second aptamer having a second configuration, wherein the aptamer second configuration is sensitive to presence of a second analyte.

Some surfaces comprise a third cluster of the plurality of clusters comprises an aptamer having a first configuration, wherein the aptamer first configuration is sensitive to presence of a first analyte.

Some surfaces comprise a third cluster of the plurality of clusters comprises a chimeric aptamer comprising at least a binding moiety of the first aptamer and at least a binding moiety of the second aptamer.

In some cases at least some of the plurality of clusters are homogenous as to aptamer composition. Alternately, in some cases at least some of the plurality of clusters are heterogeneous as to aptamer composition. Often, at least one of the plurality of clusters consists of the first aptamer, while at least one of the plurality of clusters consists of the second aptamer.

At least some of the plurality of clusters comprise a single affinity reagent such as an aptamer population per cluster in some cases. An aptamer of the aptamer clusters often comprises a detection moiety such as a fluorophore, and in some cases also comprises a quencher, or comprises a fluorophore acceptor pair.

Some surfaces are configured such that individual affinity reagents such as aptamers of a set of clusters of the plurality of clusters bind to a set of analytes implicated in a common biological process, such as a signaling pathway, for example a cancer pathway, a cancer progression evaluation pathway, a disease response pathway, a pathogen cell cycle pathway, or other disease related pathway.

Binding the first analyte to the surface often comprises delivering the analyte in an aqueous solution. Sometimes, binding the first analyte to the surface does not require processing the analyte from a sample. Alternately, some samples are processed prior to contacting to the surface, for example by enrichment, extraction, or buffering. In some cases a surface is washed subsequent to sample binding, but prior to assaying for target analyte presence. Exemplary washes include buffers, such as PBS, PBST, TBS, TBST, or others.

In various surfaces herein, the plurality of aptamer clusters comprises at least 100 clusters, such as 100, 200, 300, 400, 500, 1000, 2000, 5000, 10,000, 20,000, 50,000, 100,000, 200,000 or more than 200,000 clusters. A number of cluster sizes are consistent with the disclosure herein, for example a diameter of at least about 10 microns, 20 micros, 30 microns, 50 microns, 100 microns, 200 microns or 500 microns, among others. The plurality of aptamer clusters May exhibit a range of cluster pitches, for example at least about 20 μm, 40 μm, 60 μm, 80 μm, 100 μm, 200 μm, 500 μm or greater. The plurality of aptamer clusters exhibit a broad range of affinity reagent densities such as aptamer densities, such as about 10e14 aptamer molecules per cm2, or even 10e13, 10e12, 10e11, 10e10, 10e9, 10e8, 10e7 or less than 10e7. In some selected surface configurations, a plurality of affinity clusters such as aptamer clusters each exhibit an analyte bound conformational change at about the same temperature, such as within 0.1, 0.2, 0.5, 1, 2, 3, 4, or 5 degrees Celsius.

Similarly disclosed herein are systems for analyte detection. Some such systems comprise some or all of the following elements: a surface comprising a plurality of aptamer clusters; a surface condition modulator; and an imaging apparatus.

The system in some cases does not comprise moving parts. Alternately or in combination, some systems do not comprise a microfluidics pump or do not comprise fluid piping. The surface is in some cases an interior of a flowcell. The affinity reagent such as aptamer clusters are present at a cluster pitch of about 40 μm, or in some cases at least about 10 μm, 20 μm, 40 μm, 60 μm, 80 μm, 100 μm, 200 μm, 500 μm or greater. The affinity clusters such as aptamer clusters May 2 comprise aptamers of a common cluster that bind a common target.

In some cases the affinity clusters such as aptamer clusters comprise aptamers that are labeled, such as fluorophore labeled, and are often additionally quencher labeled, or alternately are FRET pair labeled.

The condition modulator of the systems herein regulates a conformational disruptor condition, such as temperature, ion concentration, electric field, current, voltage, vibration, sonication intensity, magnetic field or other disruptive parameter mentioned herein. In many embodiments the condition is temperature. Alternately or in combination, the condition modulator regulates a buffer condition.

A number of imaging apparatuses are consistent with the disclosure herein, such as a digital camera or a digital phone. Some imaging apparatuses are provided with the system herein, while others are independent. In some cases the imaging apparatus is fixed to the surface.

The plurality of affinity reagent such as aptamers variously comprises at least 1,000 clusters having distinct aptamer, alternately 2,000, 5,000, 10,000, 20,000, 50,000 or more than 50,000. Similarly, the plurality of aptamer clusters comprises aptamers targeting at least 1,000 distinct target analytes, alternately 2,000, 5,000, 10,000, 20,000, 50,000 or more than 50,000.

Alternately, the plurality of aptamer clusters comprises aptamers targeting at least 10 distinct target analytes, or even 5, 4, 3, 2 or a single target analyte.

The assaying is completed in no more than 5 minutes, or no more than 5, 6, 7, 8, 9, 10, 15, 20, or 30 minutes, in particular when the change in the destabilizing condition is effected gradually or incrementally, such as through subjecting the sample to a gradient. Other durations are also consistent with the disclosure herein.

Alternately, the assaying is completed in no more than 30 seconds, such as when the assaying comprises assaying at a single destabilizing condition or changing from a first to a second and optionally to a third condition parameter. Exemplary times are no more than 10, 15, 20 30 45 or 60 seconds, or no more than 1, 2, 3, 4, or 5 minutes. Other durations are also consistent with the disclosure herein.

Assaying exhibits very high sensitivity in some embodiments, such that in some cases assaying is sensitive to an analyte at a concentration of at least 1 fM, or alternately at least 10 fM, 100 fM, 1 pM, 10 pM, 100 pM, 1 nM, 10 nM, 100 nM, 1 uM or greater than 1 uM.

A number of sample types are consistent with the disclosure herein, such as a body fluid such as blood, for example in a droplet of at least 1 uL, 2 uL, 5 uL, 10 uL, 20 uL, 50 uL, or greater than 50 μL. Alternate body fluids, such as plasma, saliva, sweat, bile, urine or other fluids are similarly consistent with the disclosure herein, such as in the volumes listed.

In some selected surface configurations, a plurality of affinity clusters such as aptamer clusters each exhibit an analyte bound conformational change at about the same temperature, such as within 0.1, 0.2, 0.5, 1, 2, 3, 4, or 5 degrees Celsius. This facilitates rapid assays in some cases, as the shift to a single assay temperature or a narrow assay temperature range is sufficient to assay for a broad range of target analytes having a similar temperature at conformational change.

Also disclosed herein are methods of assaying for an analyte in a sample, comprising one or more of the following elements: contacting the sample to a surface comprising a plurality of aptamer populations, changing a condition at the surface, and assaying for a change in at least one analyte configuration.

The assaying is completed in no more than 5 minutes, or no more than 5, 6, 7, 8, 9, 10, 15, 20, or 30 minutes, in particular when the change in the destabilizing condition is effected gradually or incrementally, such as through subjecting the sample to a gradient. Other durations are also consistent with the disclosure herein.

Alternately, the assaying is completed in no more than 30 seconds, such as when the assaying comprises assaying at a single destabilizing condition or changing from a first to a second and optionally to a third condition parameter. Exemplary times are no more than 10, 15, 20 30 45 or 60 seconds, or no more than 1, 2, 3, 4, or 5 minutes. Other durations are also consistent with the disclosure herein. Often, these low assaying times are enabled by selecting affinity reagents such as aptamers having common temperature shifts for their target analyte binding.

Assaying exhibits very high sensitivity in some embodiments, such that in some cases assaying is sensitive to an analyte at a concentration of at least 1 fM, or alternately at least 10 fM, 100 fM, 1 pM, 10 pM, 100 pM, 1 nM, 10 nM, 100 nM, 1 uM or greater than 1 uM.

A number of sample types are consistent with the disclosure herein, such as a body fluid such as blood, for example in a droplet of at least 1 uL, 2 μL, 5 μL, 10 μL, 20 μL, 50 μL, or greater than 50 μL. Alternate body fluids, such as plasma, saliva, sweat, bile, urine or other fluids are similarly consistent with the disclosure herein, such as in the volumes listed.

In some selected surface configurations, a plurality of affinity clusters such as aptamer clusters each exhibit an analyte bound conformational change at about the same temperature, such as within 0.1, 0.2, 0.5, 1, 2, 3, 4, or 5 degrees Celsius. This facilitates rapid assays in some cases, as the shift to a single assay temperature or a narrow assay temperature range is sufficient to assay for a broad range of target analytes having a similar temperature at conformational change.

In some cases, the analyte comprises a protein. The method may variously distinguishes the protein according to a post-translational state of the protein, such as phosphorylation state of the protein or a glycosylation state of the protein, such as a hemoglobin glycosylation state (e g. HbAlc).

The analyte, or an analyte of the sample, may alternately or in combination comprise a small molecule, metabolite, carbohydrate, a nucleic acid, a lipid, an epitope, a cell or cellular component, a virus or a virus component, or other analyte as listed herein.

Disclosed herein are methods of assaying for an analyte in a sample, comprising one or more of the elements of contacting the sample to a surface comprising a plurality of affinity reagents such as aptamer populations, assaying for affinity reagents such as aptamer population first signal such as fluorescence, changing a condition at the surface, and assaying for affinity reagents such as aptamer population second signal such as fluorescence.

The changing of a condition in some cases comprises a gradual changing, and similarly, assaying for aptamer population second fluorescence comprises gradual assaying. In some cases changing a condition comprises a continuous changing, while in alternate cases changing a condition comprises a discrete changing.

The surface comprises wells in some cases and the aptamer populations are segregated into wells. Similarly, in some cases the surface comprises wells, and the aptamer populations are immobilized on beads, and the beads are localized into the wells. Often, the wells accommodate no more than one bead per well. To identify a bead in a particular well, the methods comprise sequencing a tag associated with a bead in a well, or sequencing an aptamer associated with a bead in a well, or both sequencing an aptamer and a tag.

Also disclosed herein are methods of distinguishing among analytes in a sample, comprising one or more of the elements of binding an unknown analyte to an affinity reagent such as an aptamer that binds a first analyte and a second analyte, changing a condition at the analyte and concurrently measuring fluorescence at the analyte, and observing a change in an output such as fluorescence, wherein the first analyte causes a change in aptamer fluorescence at a first change in the condition, and the second analyte causes a change in aptamer fluorescence at a second change in the condition. The aptamer is often tethered to a surface, such as a surface is covered with a liquid at the aptamer.

In some embodiments, the first analyte and the second analyte are proteins having identical polypeptide sequence but differ in a post-translational modification. The post translational modification is a phosphorylation or glycosylation, among other post-translational modifications disclosed herein.

The condition is often temperature, though other disrupting conditions are disclosed herein.

The aptamer often comprises a fluorophore, and often a quencher or an acceptor pair. In some cases the aptamer is immobilized in a well rather than being tethered to the surface of the array. The aptamer is in some cases tethered to a bead that is localized to a well.

The bead beneficially comprises an oligo tag, such that sequencing the oligo tag identifies the target analyte bound by the bead. Alternately or in combination, sequencing the aptamer tethered to the bead in the well identifies the target analyte bound to the bead at the position. Alternately, the tag is decoded using methods described herein or known in the art.

Disclosed herein are methods of quantifying an analyte in a sample. Some such methods comprise one or more of the elements of contacting the sample to a surface comprising a plurality of affinity clusters, wherein the affinity clusters bind the analyte in a condition-dependent manner, and assaying for first fluorescence at a first condition value.

Often, the affinity clusters comprise aptamers.

The condition governing the condition-dependent manner is often temperature, although alternatives such as voltage, current, ionic concentration, vibration, sonication or other disruptive conditions are also contemplated herein.

Some methods comprising diluting the sample prior to contacting.

Subsequent to contacting the sample to the surface, some methods comprise changing the condition, and assaying for fluorescence at a second condition value.

In some cases the number of aptamer clusters exhibiting aptamer-bound fluorescence effects quantifying the analyte in the sample Aptamers variously comprise one or more of a fluorophore, a quencher or an acceptor pair.

At least a subset of the plurality of aptamer clusters bind a common analyte in various embodiments. At least a subset of the plurality of aptamer clusters comprise distinct populations of nonidentical aptamers in various cases. The nonidentical aptamers often exhibit nonidentical analyte affinities. At least a subset of the plurality of aptamer clusters comprise aptamers having distinct surface tethering moieties, or distinct analyte binding moieties.

The aptamers having distinct surface tethering moieties may exhibit nonidentical analyte affinities. The aptamer clusters are immobilized to the surface, or are alternately immobilized by being localized to wells on the surface.

In some cases the aptamers are bound to beads, and the beads are localized to wells on the surface. The beads variously comprise bead-identifying oligo tags, that may be sequenced, for example to identify the target analyte associated with a signal arising from the affinity reagents on the bead. Alternately or in combination, beads are identified by sequencing the aptamers tethered to the beads in the well.

In light of the above, disclosed herein are systems comprising i) a surface comprising a plurality of aptamer clusters, ii) a heating unit, iii) an imager, wherein the system detects at least 100 distinct non-nucleic acid target analytes at a concentration of as low as 100 fM in no more than 10 minutes. In various embodiments, the system detects at least 1000 distinct non-nucleic acid target analytes, or at least 10,000 distinct non-nucleic acid target analytes, or at least 100,000 distinct target analytes. In various embodiments, the system detects the analytes at a concentration of as low as 10 fM or as low as 1 fM or less than 1 fM. The system detects the analytes in some cases in no more than 7 minutes, no more than 5 minutes, no more than 3 minutes, no more than 1 minute, or no more than 30 seconds.

### BRIEF DESCRIPTION OF THE FIGURES

FIG. 1 presents a schematic of analyte detection using fluorescence and environmental perturbation via heating.

FIG. 2 presents a surface cluster schematic.

FIG. 3 presents a cross-section of a sensor.

FIG. 4A presents a secondary structure of aptamers used in the examples. FIG. 4A discloses SEQ ID NO: 1.

FIG. 4B presents a secondary structure of aptamers used in the examples. FIG. 4B discloses SEQ ID NO: 3.

FIG. 4C presents a secondary structure of aptamers used in the examples. FIG. 4C discloses SEQ ID NO:

9 FIG. 5 presents solution melting curves and the first derivative of thermal titration experiments with different concentrations of T41 aptamer depicted in FIG. 4A with varying concentrations of PDGF-BB.

FIG. 6 presents dose response of PDGF-BB at different concentration of 41T aptamer (depicted in FIG. 4A) in solution.

FIG. 7 presents Solution melting curves and the first derivative of thermal titration experiments with different concentrations of HD22 aptamer depicted in FIG. 4C with varying concentrations of thrombin.

FIG. 8 presents dose response of thrombin at different concentration of HD22 aptamer (depicted in FIG. 4C) in solution.

FIG. 9A-B present normalized first derivative of the melting curves (panel A) and the calculated dose response (panel B) of the immobilized 41T aptamer depicted in FIG. 4B and varying concentrations of PDGF-BB.

FIG. 10 present normalized first derivative of the melting curves of the immobilized 41T aptamer depicted in FIG. 4B with varying concentrations of PDGF-AA. PDGF-AA is a homolog and is structurally similar to PDGF-BB.

FIG. 11 present normalized first derivative of melting curves collected in human plasma in the absence and the presence of 100 nM PDGF-BB.

FIG. 12A presents a Cross-section of a reference front-side illumination CMOS imaging sensor array.

FIG. 12B presents a cross-section of a CMOS biosensor as a representative embodiment of the invention.

FIG. 12C presents a top view of a CMOS biosensor as a representative embodiment of the invention.

FIG. 13 depicts a representative embodiment of a system comprising a biosensor mounted on a printed circuit board of a diagnostic device of this disclosure.

FIG. 14 presents a dimorphic aptamer having two binding moieties.

### DETAILED DISCLOSURE

Disclosed herein are systems, devices, compositions, and methods for the rapid detection of a large number of target analytes across a broad range of analyte concentrations in a high throughput system. Although the technology herein is compatible with antibodies and other protein-based detection moieties, some preferred embodiments rely upon target-specific aptamers that are readily synthesized using low-cost nucleic acid synthesis approaches and delivered to a surface of a chip, flowcell or bead using well-established chemical approaches. Clusters may be spotted onto surfaces or deposited into wells via beads or directly in solution at a very high density, such that a large number of analytes may be assayed for in a single reaction.

Assays are effected by observing the effect of two changes on the aptamers or other affinity reagents: firstly, a change in stability resulting from binding to target analytes, and secondly, a counteracting challenge to stability resulting from an environmental change such as an increase in temperature or other environmental challenge mentioned herein. These two changes interact such that a reporter of aptamer or other affinity reagent conformation such as a paired fluorophore system will often be stabilized upon target analyte binding. Consequently, the challenge presented by an environmental change will impact the output of such a reporter only when administered at a larger magnitude than that administered to an unbound affinity reagent.

By gradually or incrementally increasing the magnitude of an environmental change (such as an addition of heat), one can observe (or compare to previously calculated values for) the temperature or other condition at which a change in fluorescence occurs, both for bound and unbound affinity reagents. By observing a change in persistence of the fluorescence or other reporter relative to an observed or known unbound control upon incremental increases in the environmental change, one can infer binding of the target analyte to the affinity reagent.

Alternately, by selecting affinity reagents having known changes in persistence upon binding to target reagents, one can subject a set of affinity reagents to a temperature where bound and unbound affinity reagents are predicted to differentially fluoresce depending upon target analyte binding. Using this approach, one may rapidly assay for a number of target analytes using a simple environmental change regimen of one, two or three conditions.

Notably, target analyte detection is a function of the environmental condition and its impact upon the affinity reagent fluorescence. The chemistry of the target analytes is relevant only so long as the affinity reagents are able to bind them. As aptamers, for example, can be synthesized to bind to a broad range of target molecules, one can detect a broad range of target analytes using a relatively universal environmental change/reporter assay.

Accordingly, at little cost and with little time in reagent synthesis, using a common reporter assay and a common environmental change as a challenge, one may assay for a broad range of biochemically diverse target analytes, across a broad range of concentrations, in a single, rapidly executed and easily measured assay.

### Definitions

A “small molecule” is defined herein to have a molecular weight below about 1000 daltons, and is generally an organic compound. In some embodiments, a small molecule is an active agent or a prodrug or metabolite thereof. A small molecule may be charged or neutral.

The singular forms “a” “an” and “the” include plural referents unless the context clearly dictates otherwise. Approximating language, as used in the description and claims, may be applied to modify any quantitative representation that could permissibly vary without resulting in a change in the basic function to which it is related.

As used herein, the term “about” in reference to a number refers to a range spanning from 10% less than the number to 10% greater than the number. Similarly, in reference to a range, the term “about” refers to an expanded range having a lower bound which is 10% less than the lower bound listed and an upper bound which is 10% greater than the upper bound listed.

As used herein, “stabilize,” and its grammatical variants mean to hold steady or limit fluctuations. “Stabilizing” a complex results in promoting or prolonging the existence of the complex or inhibiting disruption of the complex including when the environment is changed (e.g. perturbation of the persistence of the binding competent state). The term can be applied to any of a variety of complexes including, but not limited to a binary complex. For example, the complex that is stabilized can be a binary complex between an immobilized molecule and a binding partner. Generally, stabilization of the binary complex increases the persistence of the binding competent state of the immobilized molecule upon a change in the presented environment to said immobilized molecule.

As used herein, a “nucleotide” is a molecule that includes a nitrogenous base, a five-carbon sugar (e.g., ribose, sulfo-ribose or deoxyribose), and at least one phosphate group including a phosphate ester when the nucleotide is part of a polynucleic acid, or functional analogs of such a molecule. Nucleotide analogs may optionally be without 3′-OH group, replaced with a different moiety or modified with a moiety. In some embodiments, the moiety is a 3′ hydrogen or fluorine. The base of a nucleotide may be any of adenine, cytosine, guanine, thymine, or uracil, or analogs thereof. Optionally, a nucleotide has an inosine, xanthine, hypoxanthine, isocytosine, isoguanine, nitropyrrole (including 3-nitropyrrole) or nitroindole (including 5-nitroindole) base. Nucleotides can include or exclude ATP, UTP, CTP, GTP, ADP, UDP, CDP, GDP, AMP, UMP, CMP, GMP, dATP, dTTP, dUTP, dCTP, dGTP, dADP, dTDP, dCDP, dGDP, dAMP, dTMP, dCMP, and dGMP. In some embodiments, an aptamer can comprise a nucleotide which is intrinsically a fluorophore In some embodiments, the nucleotides include SELEX-compatible nucleotides (or a variant thereof) or that can be introduced into a nucleic acid. In some embodiments, the SELEX-compatible nucleotides can include or exclude the following nucleotide modifications: substitution of 2′-OH by fluor (F), modification of 2′-OH by a methyl group (CH3), substitution of 2′-OH by an amino group (NH₂), a Locked Nucleic Acid (LNA) with methylene bridge between 2′-O and 4′-C, modification of C-5 by Bromine (Br), modification of C-5 by Iodine (I), and substitution of 4-O by Sulfur(S). In some embodiments, the SELEX-compatible nucleotides can include or exclude: 2′-Fluoro-dUTP. 2′-Fluoro-dCTP, 2′-Fluoro-dATP, 2′-Fluoro-dGTP, 2′-Fluoro-dNTP, 2′OMe-UTP, 2′OMe-CTP, 2′OMe-ATP, 2′OMe-GTP, 2′NH2-dUTP, 2′NH2-dCTP, 2′NH2-dATP, 2′NH2-dGTP, LNA-ATP, LNA-GTP, LNA-CTP, LNA-UTP, 5-Bromo-dUTP, 5-Iodo-UTP, 4-Thio-UTP, s4UTP, and 4sUTP. In some embodiments, the SELEX-compatible nucleotides can be those described in Komarova et al., Molecules. 2019 October; 24(19): 3598, herein incorporated by reference. A SELEX-compatible nucleotide can be a nucleotide which can be included in a SELEX process without negatively impacting the ability of the SELEX process to arrive at an aptamer which can selectively bind to a target.

As used herein, “measuring” (or sometimes “detecting”), refers to a process of identifying the presence of a signal. For example, measuring may involve identifying fluorescence emitted from a fluorophore upon excitation with light. Measuring can be intermittent (e.g., periodic) or continuous (e.g., without interruption), and can involve acquisition of quantitative results. Measuring can be carried out by observing multiple signals over a period of time during the changing of the environment about the fluorophore (e.g., while the temperature of the system is increased or during the application of an electric field) or, alternatively, by observing signal(s) at a single time point during or after changing the environment around the fluorophore. In some embodiments, the measuring may occur before, during, or after the change in environment. In some embodiments, measuring can be continuously monitored over time as is typical of a time-based acquisition. It is also possible to acquire a series of time points in a periodic fashion to obtain a time-based acquisition.

As used herein, “imaging” refers to a process for obtaining a representation of a sample or a portion thereof. The process may involve acquisition of optical data, such as the relative location of a feature undergoing analysis, and intensity of an optical signal produced at the position of the feature.

As used herein, “contacting,” when used in reference to chemical reagents, refers to the mixing together of reagents (e.g., mixing an immobilized molecule and either a buffered solution that may include a binding partner) so that a physical binding reaction or a chemical reaction may take place.

As used herein, “biosensor” refers to a system comprising a “receptor” and an electronic sensor. The “receptor” binds a binding partner, which may include an analyte. In some embodiments, the “receptor” is an immobilized molecule as described herein.

As used herein, “electronic sensor” refers to an electronic transducer that converts photons to electrons. In particular, an electronic sensor converts the detection of a photon or photons into an electrical signal. An electronic sensor may be electrically connected to other computer circuit units which can include or exclude memory (transitory or permanent), central processing units, and graphic processing units. In some embodiments, an electronic sensor comprises a series of stacked layers which are in electronic communication with each other and wherein at least one of the stacked layers is photosensitive. While a “sensor” refers to a device, the terms “sensor molecule”, and “biosensor molecule” refer to a modality.

In some embodiments, the immobilized molecule is a sensor molecule. Sensor molecules can be configured to be in a format where individual reactions (e.g., a binding reaction) can be isolated from another, and allows for the introduction of controlled perturbation. In some embodiments, the format can include or exclude flow cells, wells of a multiwell plate; microscope slides; tubes (e.g., capillary tubes), and beads in an emulsion. Features to be measured during changing the environment of an immobilized molecule can be contained within the isolated individual reactions. In some embodiments, the sensor molecule is connected to a solid support. In some embodiments, the sensor molecule is directly connected to a solid support by a covalent or non-covalent bond. In some embodiments, the sensor molecule is linked to the solid support by a linker. The linker can comprise a polymer. In some embodiments, the polymer is a hydrogel.

As used herein, the term “solid support” refers to a rigid substrate that is insoluble in an aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, beads, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, 2D materials (e.g., graphene), transparent conductive materials (e.g., Indium Tin Oxide, silver nanoparticle doped polymers), metals, inorganic glasses, mirrored surfaces, and polymers.

The solid support may take any of a variety of configurations ranging from simple to complex and can have any one of a number of shapes, including a strip, plate, disk, rod, particle, including bead, tube, well, and the like. The surface may be relatively planar (e.g., a slide), spherical (e.g., a bead), cylindrical (e.g., a rod), or grooved. Exemplary solid supports that may be used can include or exclude microtiter wells, microscope slides, membranes, paramagnetic beads, charged paper, Langmuir-Blodgett films, silicon wafer chips, flow through chips, and microbeads.

As used herein, “library” refers to a collection of species wherein not all of the species have the same identity.

As used herein, a “complex” refers to a molecular entity formed by covalent or non-covalent association involving two or more component molecular entities (e.g., an immobilized molecule and a binding partner) The complex is not necessarily transitory, in that the complex will remain as a bimolecular entity until subject to a change in environment. The complex forms because of biomolecular recognition between the immobilized molecule and the binding partner.

As used herein, “equilibrium” generally refers to a state of balance due to the equal action of opposing forces (e.g., equal, opposite rates). For example, a complex formed between an immobilized molecule and a binding partner is in equilibrium with unbound immobilized molecule and binding partner when the rate of formation of the complex is balanced by the rate of its dissociation. Under this condition, the reversible binding reaction ceases to change its ratio of bound/unbound component. If the rate of a forward reaction (e.g., complex formation) is balanced by the rate of a reverse reaction (e.g., complex dissociation), then there is no net ratio change.

As used herein, “binding competent state” refers to the conformation or ensemble of conformations that an immobilized molecule adopts which can form a complex with a binding partner. In some embodiments, the conformation is a tertiary structure of the immobilized molecule (“binding competent conformation”). The binding competent state need not have a bound binding partner, but is a local energy minimum based on the structure of the immobilized molecule. In some embodiments, when the immobilized molecule is an aptamer, the binding competent state is the tertiary structure of the aptamer formed from the local energy minimum for folding. In some embodiments, aptamers in a binding competent state may comprise regions of double-strands, hairpin loops, and/or single strand sequences. In some embodiments, a cluster of immobilized molecules can comprise ensembles of binding competent states. For example, a first immobilized molecule in a cluster can comprise a first binding competent state, and a second immobilized molecule within the same cluster and having the same identity of the first immobilized molecule can comprise a second binding competent state. In another example, a cluster can be formed from two different aptamers that each recognize different parts of the same analyte, such that avidity favors the selective binding of the analyte. Complex formation can involve conformational changes as a prerequisite for binding (e.g., conformational selection) or concurrently with binding (e.g., induced-fit). Regardless, the stability/persistence of the binding competent ensemble of states will change upon association with a binding partner.

The term “sample” as used herein refers to an aliquot of material, frequently an aqueous solution or an aqueous suspension derived from biological material. In some embodiments, the sample can be a biological sample. The biological sample can be from a living subject. For example, in some embodiments, the sample may be any sample containing cells. In some embodiments, the sample may be from, for example, whole blood, bone marrow, serum, plasma, cerebrospinal fluid, sputum, bronchial washings, bronchial aspirates, urine, lymph fluids and various external secretions of the respiratory, intestinal and genitourinary tracts, tears, saliva, sweat, milk, white blood cells, myelomas and the like; biological fluids such as cell culture supernatants, tissue specimens which may or may not be fixed, and cell specimens which may or may not be fixed, or a fine needle aspirate. Samples to be assayed for the presence of an analyte by the methods of the present invention include, for example, cells, tissues, homogenates, lysates, extracts, purified or partially purified proteins and other biological molecules and mixtures thereof. In some embodiments, the biological sample may be processed. The processing can be, for example, removal of selected species in the sample.

As used herein, “subject” refers to any source of biological or nonbiological sample for which an analyte is to be assayed. In some cases the subject is a mammal that can include or exclude humans, domestic and farm animals, and zoo, or pet animals, such as dogs, horses, cats, mouse, rat, rabbits, monkeys, llama, sheep, pigs, cows, etc., or exotic or ‘wild’ animals such as bats, wild animals caught for sale, or wild animals (for example, one suspected of harboring a pathogen capable of impacting humans). The preferred mammal herein is a human, including adults, children, and the elderly. In some embodiments, the subject is an aquatic park animal, such as a dolphin, whale, seal or walrus. A subject can also include any organism used in clinical or preclinical trials. Alternately, a subject may comprise an environmental sample, such as an environmental sample suspected of harboring an organism or analyte of interest.

A sample drawn from a subject may be assayed subsequent to processing, such as buffering, stabilization, or purification of the sample by, for example, purification or enrichment of proteins, small molecules such as hormones or starches, nucleic acids such as DNA or RNA, or lipids, cells, viruses, or other molecule or molecule classes in the sample, removal of proteins, small molecules such as hormones or starches, nucleic acids such as DNA or RNA, or lipids, cells, viruses, or other molecule or molecule classes from the sample. Alternately, a sample may be assayed raw, without subjecting the sample to selective enrichment, buffering or stabilization. Furthermore, subsequent to contacting a sample to a binding agent, the sample and binding agent, surface, beads or other binding vicinity may be subjected to a wash step. The wash step may serve to remove constituents of the sample that may interfere with the assay, by for example blocking binding or leading to background fluorescence that may impact signal detection, or may remove nonspecifically bound sample constituents. Other wash functions are consistent with the disclosure herein.

As used herein, the term “binding partner” refers to any known or unknown substance that can be recognized by the immobilized molecule. The term “binding partner” may include, for example, ions, small molecules (e.g., having a molecular weight of less than 1000 Da), proteins, peptides, glycoproteins, cells, cell-surface molecules or proteins or glycoproteins, viruses, organelles, synthetic polymers, carbohydrates, hormones, cytokines, growth factors, toxins, cell surface receptors, bacterial or parasitic cell components, or viral antigens, or a component thereof In some embodiments, a binding partner may be obtained from a sample comprising complex mixture of ions, small molecules (e.g., having a molecular weight of less than 1000 Da), proteins, peptides, glycoproteins, a cell, a cell-surface molecule or protein or glycoprotein, a virus, an organelle, a synthetic polymer, a carbohydrate, or a component thereof. In some embodiments, when the binding partner is a cell, the cells may be a transformed cell which can be transfected with an oncogene which is integrated into the cell. In some embodiments, the transformed cells may include or exclude, for example, mammalian cells, immunomodulatory cells, leukocytes, tumor cells, yeast cells, bacterial cell, infectious agents, parasites, plant cells, transfected cells such as NSO, CHO, COS, 293 cells. Transformation of cells such as NSO, CHO, COS and 293 cells can be achieved by a method which can include or exclude electroporation and nucleofection. In some embodiments, the binding partner can be present on the cell surface, within the cell, or both on the surface and within the cell. In some embodiments the binding partner May be present in or on one or more cellular features, for example, the cytosol, the nucleus, the nuclear membrane, nucleoli, the endoplasmic reticulum, Golgi apparatus or mitochondria.

In some embodiments, both the binding partner and the immobilized molecule are not nucleic acids (e.g., DNA microarray). When the binding partner is a nucleic acid, the immobilized molecule is often not a nucleic acid. When the immobilized molecule is a nucleic acid, the binding partner is often not a nucleic acid. The terms “specifically binding” and “specific binding” as used herein mean that a protein (e.g., antibody or lectin), aptamer, or other immobilized molecule of interest, binds to a target such as an antigen, ligand or other analyte, with a different affinity than it binds to other molecules under the specified conditions of the present invention, such that under certain conditions the molecule of interest can be said to be specifically bound while other molecules are not bound by the binding moiety.

As used herein, “environment” refers to the surroundings or conditions to which the immobilized molecule is exposed. An environment may include or exclude contributions from an electric field, a magnetic field, a thermal energy, a gravitational field, flow rate, shear rate, light intensity or wavelength or polarization, ionic strength, or chaotropic agent concentration. In some embodiments, one element of an environment may be present while the others are absent. For example, the ionic strength may be a selected buffer concentration but the chaotropic agent concentration is zero. An environment may be changed by the application of an external force. The applied external force can include or exclude: electric field intensity, electric field direction, magnetic field, gravitational field, shear force, increased or decreased temperature, light polarization change, light intensity change, light wavelength change, increase or decrease in an ion or chaotropic agent concentration. In some embodiments, an environment change may result in the denaturation of the immobilized molecule. In some embodiments, an environmental change may result in the folding or unfolding of an immobilized molecule (e.g., protein or nucleic acid). In some embodiments, the environmental change may result in a conformational change of the immobilized molecule.

As used herein, the term “chaotropic agent” includes its commonly understood meaning in the field and refers to agents such as guanidinium hydrochloride or urea, which disrupt hydrogen bonds to potentially destabilize the binding competent states of biomolecules.

As used herein, the term “electric field” refers to a field generated by the presence of a voltage gradient which exerts a force on a point charge or a force on a multipole. The intensity of an electric field can be modulated resulting in a corresponding change in the applied force upon a charged particle or multipole subject to the electric field. An electric field is a vector, in that there is also a directional element. In some embodiments, the magnitude and/or direction of an electric field may be changed to perturb the environment upon which an immobilized molecule is subject to.

As used herein, a “kit” is a packaged unit containing one or more components that can be used for performing detection of analytes. Typical kits may include packaged combinations, in one or more containers or vials of reagents, a consumable cartridge, configured to be used in the methods described herein.

As used herein, the term “antibody” refers to an immunoglobulin or fragment thereof that can specifically bind to an antigen (binding partner). In some embodiments, an antibody can include or exclude any recombinant or naturally occurring immunoglobulin molecule such as a member of the IgG class, (e.g., IgG1), antibody fragment, ScFv (single-chain variable fragment), a fusion protein of the variable regions of the heavy (VH) and light chains (VL) of immunoglobulins (optionally wherein the fusion VH and VL chains are connected with a short linker peptide of ten to about 25 amino acids), or single-domain antibody (nanobody), and any derivatives thereof. In some embodiments, the antibody can be a monoclonal or polyclonal antibody.

The term “antibody fragments” as used herein, refers to a portion of an intact antibody, wherein the portion retains at least one, and as many as most or all, of the functions normally associated with that portion when present in an intact antibody. In some embodiments, an antibody fragment comprises an antigen binding site of the intact antibody and thus retains the ability to bind an antigen. An antibody fragment can include or exclude Fv, Fab and F(ab′)2 fragments.

“Polyclonal Antibodies” or “PAbs,” are heterogeneous populations of antibody molecules derived from the sera of animals immunized with an antigen, or an antigenic functional derivative thereof. For the production of polyclonal antibodies, host animals such as rabbits, mice and goats, may be immunized by injection with an antigen or antigen-conjugate, optionally supplemented with adjuvants. Polyclonal antibodies may be unpurified, purified or partially purified from other species in an antiserum. The techniques for the preparation and purification of polyclonal antibodies are described in various general and more specific references, including but not limited to Kabat & Mayer, Experimental Immunochemistry, 2d ed., (Thomas, Springfield, Ill. (1961)); Harlow & Lane, Antibodies: A Laboratory Manual (Cold Spring Harbor Laboratory Press, Cold Spring Harbor, N.Y. (1988)); and Weir, Handbook of Experimental Immunology, 5th ed. (Blackwell Science, Cambridge, Mass. (1996)).

“Monoclonal antibodies,” or “MAbs,” are homogeneous populations of antibodies to a particular antigen and may be obtained by any technique that provides for the production of antibody molecules, such as by continuous culture of cell lines. These techniques include, but are not limited to the hybridoma technique of Köhler and Milstein, Nature, 256:495-7 (1975); and U.S. Pat. No. 4,376,110), the human B-cell hybridoma technique (Kosbor, et al., Immunology Today, 4:72 (1983); Cote, et al., Proc. Natl. Acad Sci. USA, 80:2026-30 (1983)), and the EBV-hybridoma technique (Cole, et al., in Monoclonal Antibodies And Cancer Therapy, Alan R. Liss, Inc., New York, pp. 77-96 (1985)). Such antibodies may be of any immunoglobulin class including IgG, IgM, IgE, IgA, IgD and any subclass thereof. The hybridoma producing the MAb of this invention may be cultivated in vitro or in vivo. Production of high titers of MAbs in vivo makes this a presently preferred method of production.

Techniques described for the production of single chain antibodies (U.S. Pat. No. 4,946,778; Bird, Science 242:423-26 (1988); Huston, et al., Proc. Natl. Acad. Sci. USA, 85:5879-83 (1988); and Ward, et al., Nature, 334:544-46 (1989)) can be adapted to produce single chain antibodies suitable for use in the present invention. Single chain antibodies are typically formed by linking the heavy and light chain fragments of the Fv region via an amino acid bridge, resulting in a single chain polypeptide.

Antibody fragments include but are not limited to: the F(ab′)2 fragments that can be produced by pepsin digestion of the antibody molecule and the Fab fragments that can be generated by reducing the disulfide bridges of the F(ab′)2 fragments. Alternatively, Fab expression libraries may be constructed (Huse, et al., Science, 246:1275-81 (1989)) to allow rapid and easy identification of monoclonal Fab fragments with the desired specificity.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Techniques developed for the production of “chimeric antibodies” (Morrison, et al., Proc. Natl. Acad. Sci., 81:6851-6855 (1984); Takeda, et al., Nature, 314:452-54 (1985)) by splicing the genes from a mouse antibody molecule of appropriate antigen specificity together with genes from a human antibody molecule of appropriate biological activity can be used. A chimeric antibody can be a molecule in which different portions are derived from different animal species, such as those having a variable region derived from a murine MAb and a human immunoglobulin constant region.

The terms “polynucleotide” and “nucleic acid” are used interchangeably to refer to polymeric forms of nucleotides of any length. The polynucleotides may comprise deoxyribonucleotides, ribonucleotides and/or their analogs. Polynucleotides may have any three-dimensional structure, and may perform any function, known or unknown. A nucleic acid molecule may also comprise modified nucleotides, such as methylated nucleotides and nucleotide analogs. Analogs of purines and pyrimidines are known in the art, and include, but are not limited to, aziridinycytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, S-methylcytosine, pseudouracil, 5-pentylnyluracil and 2,6-diaminopurine. The use of uracil as a substitute for thymine in a deoxyribonucleic acid is also considered an analogous form of pyrimidine.

Sugar modifications (e.g., 2′-O)-methyl, 2-fluor and the like) and phosphate backbone modifications (e.g., morpholino, locked nucleic acid (LNA), unlocked nucleic acid, peptide nucleic acid (PNA), thioates, dithioates, phosphorothiolates, phosphorothioates, methyl phosphonates, and the like) can be incorporated singly, or in combination, into the nucleic acid molecules of the present invention. In some embodiments, for example, a nucleic acid of the invention may comprise a modified sugar and a modified phosphate backbone. In another embodiment, a nucleic acid of the invention may comprise modifications to sugars, bases, and/or phosphate backbone.

The nucleotide sequence of the aptamer nucleic acids of the present invention is of less importance than the functional roles they are required to perform. Accordingly, the sequence of the aptamer nucleic acids, and the length of the aptamer nucleic acid, may vary considerably, provided the aptamer nucleic acid can still perform the functional roles they are required to perform. Importantly, the sequence and length of the aptamer nucleic acids are not limited to those exact sequences and lengths of the exemplary binding pairs disclosed herein. The aptamer nucleic acids thus can be of different lengths and or sequence, and vary in identity and/or length to the disclosed aptamer nucleic acids. In some embodiments, the aptamer nucleic acids can have 80, 85, 88, 90, 91, 92, 93, 94, 95, 96, 97, 98, or 99% identity to those aptamer sequences disclosed herein. An important function of the aptamer nucleic acid of the present invention is to provide a binding competent state to the binding partner to form a complex.

As used herein, “aptamer” refers to a nucleic acid that binds to a target (a binding partner). In some embodiments, the binding is specific binding for a target molecule, such target molecule having a three-dimensional chemical structure, other than a polynucleotide, that binds to the aptamer through a mechanism which is predominantly independent of Watson/Crick base pairing or triple helix binding. In some embodiments, the aptamer can be one such as those described in S. Lapa, et al., Molecular Biotechnology volume 58, p. 79-92 (2016); or S. Gao, et al., Analytical and Bioanalytical Chemistry volume 408, p. 4567-4573 (2016)), herein incorporated by reference. Aptamers include nucleic acids that are identified from a candidate mixture of nucleic acids by the process referred to as SELEX and variations thereof. In some embodiments, the aptamer is developed by a method comprising: (a) contacting the candidate mixture with the target, wherein nucleic acids having an increased affinity to the target relative to other nucleic acids in the candidate mixture may be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers to the target molecule are identified. Affinity interactions May vary in degree; however, in this context, the “specific binding affinity” of an aptamer for its target means that the aptamer binds to its target generally with a higher degree of affinity than it may binds to other, non-target, components in a mixture or sample. A “cluster of aptamers” is a set of copies of one type or species of nucleic acid molecule that has a particular nucleotide sequence and are grouped together in a defined location on a surface. An aptamer can include any suitable number of nucleotides. Different aptamers may have either the same number or a different number of nucleotides. Aptamers may comprise or be DNA or RNA or variants thereof, and may comprise single stranded, double stranded, and/or hairpin regions.

In some embodiments, the nucleic acid composition of an aptamer can be varied to produce an aptamer with a selected persistence upon self-folding into a binding competent state or to optimize its affinity for its target. In some embodiments, the modified nucleic acid in the aptamer can include or exclude: peptide-nucleic acids (PNA), locked nucleic acids (LNA), or normal deoxyribonucleic acid (DNA). PNAs have a peptide-backbone rather than a ribose-phosphate backbone of normal DNA. The PNA backbone is composed of repeating N-(2-aminoethyl)-glycine units linked by peptide bonds. The purine and pyrimidine bases are linked to the PNA backbone by a methylene bridge (—CH₂—) and a carbonyl group (—(C═O)—), The PNA backbone thus lacks charged phosphate groups. PNAs are not easily recognized by either native nucleases or proteases, imbuing them resistance to enzymatic degradation and pH stability. The LNA backbone comprises a ribose moiety which is modified with an extra bridge connecting the 2′ oxygen and 4′ carbon locking the ribose in 3′-endo (North) conformation. The locked ribose conformation enhances base stacking and backbone pre-organization and significantly increases the duplex stability of LNA/DNA duplexes. Methyl phosphonate backbones replace the charged anionic phosphate with a neutral methyl phosphonate ester. Thiophosphonate backbones comprise non-bridging oxygen on the phosphate backbone to form a phosphorothioate (PS) linkage. Thiophosphonate backbones exhibit nuclease resistance should a nuclease be present in the sample. Not all of these constituent nucleotides can be included in SELEX, but can be used to modify a nucleic acid molecule.

In some embodiments, the aptamer can further comprise one or a plurality of non-natural nucleotides. In some embodiments, the non-natural nucleotide can be iso-G or iso-C (or derivatives thereof), as described in Richert, C., et al. J. Am. Chem. Soc. 118, 4518-4531 (1996), herein incorporated by reference. In some embodiments, the non-natural nucleotide can be diflurotoluene (or derivatives thereof), as described in Schweitzer, B. A., et al., J. Am. Chem. Soc. 117, 1863-1872 (1995), herein incorporated by reference. In some embodiments, the non-natural nucleotide can be MMO2 or SICS (or derivatives thereof), as described in Leconte, A. M. et al. J. Am. Chem. Soc. 130, 2336-2343 (2008), herein incorporated by reference. In some embodiments, the non-natural base can be Ds or Diol-Px (or derivatives thereof), as described in Yamashige, R. et al. Nucl. Acids Res. 40, 2793-2806 (2012), herein incorporated by reference. In some embodiments, the non-natural nucleotide can be P or Z (or derivatives thereof), as described in Yang; Z., et al., J. Am. Chem. Soc. 133, 15105-15112 (2011), herein incorporated by reference. In some embodiments, the non-natural nucleotide can be NaM or 5SICS (or derivatives thereof), as described in Malyshev, D. A. et al. Proc. Natl Acad. Sci. USA 109, 12005-12010 (2012), herein incorporated by reference. In some embodiments, the non-natural nucleotide can be of an expanded genetic code as described in Kimoto et al., Chem. Soc. Rev., 2020, 49, 7602-7626, herein incorporated by reference. In some embodiments, the aptamers comprising one or a plurality of non-natural nucleotides can be modified before, or after SELEX identification so as to include a non-natural nucleotide.

In some embodiments, the chemistry methods used to covalently connect the biomolecule (nucleic acid, antibody, protein, or peptide) to a dye can be accomplished by the bioconjugation methods described in Hermanson, G., Bioconjugate Techniques, Academic Press (1996), herein incorporated by reference in its entirety.

As used herein, “immobilized molecule” refers to a molecule which is located at a particular region such that it may be located subsequent to detection. In some embodiments, a molecule is immobilized as a result of it being connected to a solid support such that the immobilized molecule may not translocate on or off the solid support. In some embodiments, the immobilized molecule is a synthetic polymer or a biomolecule (“immobilized biomolecule”). Examples of the immobilized biomolecule can include or exclude an ion, a small molecule, antibody, aptamer, protein, lectin, aptamer, carbohydrate (e.g., sugar or oligosaccharide), or peptide. In some embodiments, the immobilized biomolecule includes an immobilized biosensor biomolecule (immobilized sensor biomolecule).

Alternately, in some cases a molecule is immobilized by being localized to a confined volume, such as a well or an emulsion droplet. In these cases the molecule is not tethered but is located at a particular region such that it may be located subsequent to detection.

In some embodiments, the immobilized molecule comprises one or more covalently attached dyes (including a fluorescent dye). For example, the dye can be chemically linked to the immobilized protein using a cysteine or lysine residues (naturally present or available via mutations) or to the free amino group of the N-terminus. Exemplary fluorophores include, but are not limited to, fluorescent nanocrystals;

quantum dots; d-Rhodamine acceptor dyes including dichloro[R110], dichloro[R6G], dichloro[TAMRA], dichloro[ROX] or the like; fluorescein donor dye including fluorescein, 6-FAM, or the like; Cyanine dyes such as Cy3B; Alexa dyes, SETA dyes, Atto dyes such as atto 647N which forms a FRET pair with Cy3B and the like. Fluorophores include, but are not limited to, MDCC (7-diethylamino-3-[([(2-maleimidyl)ethyl]amino)carbonyl]coumarin), FAM, TET, HEX, Cy3, Cy3B, TMR, ROX, Texas Red, TAMRA, Cy5, Cy7, Cy3.5, Cy7.5, LC red 705 and LC red 640. Fluorophores and methods for their use including attachment to antibodies and other molecules are described in The Molecular Probes® Handbook (Thermo Fisher, Carlsbad, California) and Fluorophores Guide (Promega, Madison, Wisconsin), which are incorporated herein by reference in their entireties. Exemplary quenchers include, but are not limited to, ZEN, IBFQ, BHQ-1, BHQ-2, DDQ-I, DDQ-11, Dabcyl, Qx1 quencher, Iowa Black RQ, and IRDye QC-1. In some embodiments, the aptamer comprises a nucleotide which is a fluorophore.

In some embodiments, the immobilized molecule is an aptamer which is not covalently labeled. In such an embodiment, the aptamer is further complexed with an intercalating dye. The intercalating dye exhibits fluorescence when in the presence of a binding competent state which includes hybridized segments. When the environmental change induces a change in the aptamer or other affinity reagent structure such that it becomes unfolded or otherwise changes its conformation (so as to lose the persistence of the original binding competent state), the fluorescence signal decreases. The aptamer persistence loss measured with an intercalating dye can be performed in both the presence and absence of a binding partner to compare the difference, per the methods described herein. The intercalating dye can be an intercalating dye disclosed in U.S. Pat. No. 8,399,196, herein incorporated by reference. The intercalating dye can be selected from: DAPI (4′,6-diamidino-2-phenylindole), 7-AAD (7-aminoactinomycin D), ethidium bromide, Hoechst 33258 (4-[6-(4-methyl-1-piperazinyl) [2,6′-bi-1H-benzimidazol]-2′-yl]-phenol, trihydrochloride) (and also 33342, 34580), YOYO-1/DiYO-1/TOTO-1/DiTO-1 (YOYO-1 is also referred to as [12(2)Z, 16(172)Z]-13,7,7,11,11,173-Hexamethyl-13H,173H-7,11-diaza-31λ5,151λ5-3(4,1),15(1,4)-diquinolina-1,17(2)-bis([1,3]benzoxazola)heptadecaphane-12(2),16(172)-diene-7,11-diium-31, 151-bis(ylium)tetraiodide), Sybr Gold ([2-[N-(3-dimethylaminopropyl)-N-propylamino]-4-[2,3-dihydro-3-methyl-(benzo-1,3-thiazol-2-yl)-methylidene]-1-phenyl-quinolinium]), and Sybr Green (N′,N′-dimethyl-N-[4-[(E)-(3-methyl-1,3-benzothiazol-2-ylidene)methyl]-1-phenylquinolin-1-ium-2-yl]-N-propylpropane-1,3-diamine), and any other DNA intercalating dye referenced in the Molecular Probes catalog identified herein.

In some embodiments, a conformationally sensitive dye may be attached close to the region of the immobilized molecule which forms a complex with the binding partner to increase its sensitivity to complex formation. In some embodiments, a solvatochromic dye may be employed to monitor conformational transitions in the immobilized molecule; wherein the change in local polar environment induced by the conformational change can be used as the reporter signal. Solvatochromatic dyes can include or exclude: Reichart's dye, IR44, merocyanine dyes (e.g., merocyanine 540), 4-[2-N-substituted-1,4-hydropyridin-4-ylidine)ethylidene]cyclohexa-2,5-dien-1-one, red pyrazolone dyes, azomethine dyes, indoaniline dyes, diazamerocyanine dyes, and indigoid dyes, as exemplified by indigo. Methods to introduce dyes or fluorophores to specific sites of an immobilized molecule can be performed by the bioconjugation chemistries described herein.

In some embodiments, the immobilized molecule is labeled with a dye pair wherein each dye is at a position on the immobilized molecule that is sensitive to the conformational of the competent binding state of the immobilized molecule. In some embodiments, when the immobilized molecule is a protein, the protein may be a native or modified protein. Modified proteins include those with one or more amino acid mutations, additions, and/or deletions. Optionally, one or more, but not all, cysteine or lysine amino acids are mutated to another amino acid, such as alanine. In this case, the remaining one or more cysteines or lysines are used for site-specific conjugation to a dye (e.g., fluorophore). Alternatively, one or more amino acids are mutated to a reactive amino acid suitable for fluorophore conjugation, such as cysteines or lysines. In some embodiments, the amino acid can be a non-canonical amino acid. The non-canonical amino acids can be introduced for dye conjugation according to methods described in the literature (see Saleh, et al. Non-canonical amino acid labeling in proteomics and biotechnology. J Biol Eng 13, 43 (2019), doi.org/10.1186/s13036-019-0166-3 Acevedo-Rocha et al., DOI: 10.1039/C3CY20712A (Communication) Catal. Sci. Technol., 2013, 3, 1198-1201; Fang et al., (2018) Incorporation of Non-Canonical Amino Acids into Proteins by Global Reassignment of Sense Codons In: Udit A. (eds) Protein Scaffolds. Methods in Molecular Biology, vol 1798. Humana Press, New York, NY, doi.org/10.1007/978-1-4939-7893-9_13; Leisle L, Valiyaveetil F, Mehl RA, Ahern CA. Incorporation of Non-Canonical Amino Acids Adv Exp Med Biol. 2015; 869:119-151. doi: 10.1007/978-1-4939-2845-3_7; and Edward A. Lemke, Ed., Noncanonical Amindo Acids, Springer Protocols, ISBN: 978-1-4939-7574-7, doi.org/10.1007/978-1-4939-7574-7), all of which are herein incorporated by reference. Non-canonical amino acids can be commercially sourced (e.g., Bio-techne/Tocris).

By way of example, the immobilized molecule may comprise a fluorescent dye. To measure the immobilized molecule-binding partner complex formation with high signal-to-noise, evanescent illumination, confocal imaging, or off-alignment excitation may be employed. The persistence of a binding competent state (or ensemble of states) of a dual-labelled aptamer, for example, may be measured as an increased fluorescence compared to the background, for instance, upon subjecting to an environmental change, whereas in some instances it may be also be observed as a decreased fluorescence due to quenching, change in local polar environment or change in local stability at the site of the fluorophore pair despite an overall increase in stability of the aptamer. In some embodiments, a “cluster” (a clonal population of immobilized molecules all having the same identity and immobilized within a single spot) may be attached to a support surface such as an essentially planar substrate, microparticle, or nanoparticle. Optionally, an immobilized molecule is labelled with dye selected from a fluorophore, luminophore, chemiluminophore, chromophore, or bioluminophore.

In some embodiments, the dye or dye pair can be chemically linked to the immobilized molecule (e.g., antibody or protein) using a free sulfhydryl or a free amine moiety of the immobilized molecule. This can involve chemical linkage to the immobilized molecule (e.g., antibody or protein) through the side chain of a cysteine residue, through the free amino group of the N-terminus (or cleaved N-terminus for native chemical ligation). A dye can also be attached to the immobilized molecule (e.g., antibody or protein) via protein fusion. Exemplary fluorescent dyes that can be attached via protein fusion include, for example, fluorescent proteins (e.g., Green Fluorescent Protein, and wavelength shifted variants thereof including mNeonGreen, CFP, YFP, etc.) and phycobiliproteins (e.g., phycocyanin, phycoerythrin and variants thereof).

In some embodiments, when the dye is a fluorophore, the fluorophore can comprise a fluorescent moiety and conjugation moiety. Exemplary fluorescent moieties can include the dyes described herein and further can be selected from: rhodols; resorufins; coumarins; xanthenes; acridines; fluoresceins; rhodamines; erythrins; cyanins; phthalaldehydes; naphthylamines; fluorescamines; benzoxadiazoles; stilbenes; pyrenes; indoles; borapolyazaindacenes, quinazolinones; eosin; erythrosin; Malachite green; CY dyes (GE Biosciences), including Cy3 (and its derivatives) and Cy5 (and its derivatives); DYOMICS and DYLIGHT dyes (Dyomics) including DY-547, DY-630, DY-631, DY-632, DY-633, DY-634, DY-635, DY-647, DY-649, DY-652, DY-678, DY-680, DY-682, DY-701, DY-734, DY-752, DY-777 and DY-782; Lucifer Yellow; CASCADE BLUE; TEXAS RED; BODIPY (boron-dipyrromethene) (Molecular Probes) dyes including BODIPY 630/650 and BODIPY 650/670; ATTO dyes (Atto-Tec) including ATTO 390, ATTO 425, ATTO 465, ATTO 610 611X, ATTO 610 (N-succinimidyl ester), ATTO 635 (NHS ester); ALEXA FLUORS including ALEXA FLUOR 633, ALEXA FLUOR 647, ALEXA FLUOR 660, ALEXA FLUOR 700, ALEXA FLUOR 750, and ALEXA FLUOR 680 (Molecular Probes); DDAO (7-hydroxy-9H-(1,3-dichloro-9,9-dimethylacridin-2-one or any derivatives thereof) (Molecular Probes); QUASAR dyes (Biosearch); IRDYES dyes (LiCor) including IRDYE 700DX (NETS ester), IRDYE 80016 (NETS ester) and IRDYE 800CW (NETS ester); 29 EVOBLUE dyes (Evotech Biosystems); JODA 4 dyes (Applied Biosystems); HIL YTE dyes (AnaSpec); MR121 and MR200 dyes (Roche); Hoechst dyes 33258 and 33242 (Invitrogen); FAIR OAKS RED (Molecular Devices); SUNNYVALE RED (Molecular Devices); LIGHT CYCLER RED (Roche); EPOCH (Glen Research) dyes including EPOCH REDMOND RED (phosphoramidate), EPOCH YAKIMA YELLOW (phosphoramidate), EPOCH GIG HARBOR GREEN (phosphoramidate); Tokyo green (M. Kamiya, et al., 2005 Angew. Chem. Int. Ed. 44:5439-5441); and CF dyes including CF 647 and CF555 (Biotium). Exemplary conjugation moieties are chemical handles which can form covalent bonds with the biomolecule and can include or exclude: NHS (N-hydroxy-succinimide), azide, tetrazine, alkyne (including strained alkyne, such as dibenzocyclooctyne group (DBCO as described in the example 3)), aldehyde, oxo-amine, imine-formation moieties (e.g., Solulink Hydrazine), maleimide, thiol, amine, and alkyl halide. In some embodiments, the fluorophore can further comprise a spacer moiety. The spacer moiety can include or exclude a polyethylene glycol polymer (for example, with 1 to 20 repeat units), polypropylene glycol polymer (for example, with 1 to 20 repeat units), polyethylene or polypropylene polymer (for example, with 1 to 20 repeat units).

As used herein, “energy transfer relationship” refers to a relationship between two dyes (e.g., a “donor” and an “acceptor”) held sufficiently close that energy emitted by one dye can be received or absorbed by the other dye. The “donor” is the moiety that initially absorbs the energy, and the “acceptor” is the moiety to which the energy is subsequently transferred.

As used herein, “FRET” (i.e., Forster resonance energy transfer) refers to the distance-dependent transmission of energy from the site of absorption to the site of its utilization (e.g., fluorescence) in a molecule or system of molecules by resonance interaction between chromophores.

As used herein, “persistence” refers to the state of occurring or existing under conditions different from, such as beyond the usual, expected, or normal time. The increased persistence of a binding competent state of an immobilized molecule refers to the continued or prolonged existence of the binding competent state. An immobilized molecule may have a binding competent state in a first environmental condition. Changing the immobilized molecule environment to a second environmental condition may change the stability of the binding competent state, resulting in the increase, reduction, or complete loss of persistence of the binding competent state. In some embodiments, increased persistence of a binding competent state refers to an increased stability of a tertiary conformation or ensemble of conformations of an immobilized molecule.

In some embodiments, the persistence of the immobilized molecule can be measured as a function of a change of environment by using one or a plurality of dye pairs attached to the immobilized molecule. The dye may be monitored by detection methods including, but limited to, optical, electrical, thermal, mass, size, charge, anisotropy, and vibration. Rotation and vibration of a molecule can be measured using Raman and/or Infrared (IR) spectroscopy. Mass and size may be measured by surface plasmon resonance (SPR), atomic force microscopy (AFM), or scanning tunneling microscopy (STM). Persistence may be monitored continuously or at selected intervals, as the environment changes. The interval of monitoring can vary between microseconds to hours.

Fluorescence of the dye attached to the aptamer often serves as a proxy reporter for overall aptamer stability, such that increased fluorescence relative to an unbound control upon subjecting an aptamer to an environmental change is indicative of increased persistence consistent with target analyte binding. However, in some cases target analyte binding, while increasing stability of the aptamer complex, may locally disrupt fluorophore or other reporter activity. In these cases a decrease in reporter activity indicates target analyte binding. Nonetheless, the change in reporter activity in response to a change in environmental conditions such as a disruptive treatment is indicative of target analyte binding to the aptamer or other affinity reagent.

In some embodiments, persistence of a binding competent state or ensemble of states can be measured by detecting an increase in fluorescence upon destabilization of a binding competent state (e.g., unfolding of the aptamer), wherein the dye pair is a fluorophore/quencher pair and when the distance between the fluorophore and quencher is increased by a change in the 21 environment (e.g., application of thermal energy or an electric field or other disruptive factor 22 disclosed herein or known in the art), formerly quenched fluorescence appears. In some embodiments, persistence of a binding competent state can be measured by detecting a decrease in fluorescence upon loss of the stabilizing conformation, wherein the dye pair is a donor/acceptor pair and when the distance between the donor and acceptor is increased by a change in the environment (e.g., application of thermal energy or an electric field or other disruptive factor disclosed herein in known in the art) leading to the loss of the stabilizing confirmation and of the formerly high fluorescence emitted by the acceptor is incrementally quenched due to the change in Förster distance between the donor and acceptor. The fluorescent signals may be used to monitor the kinetics the change in aptamer conformation when the fluorescence is continuously measured as the environment changes, and when discrete environmental changes are applied, fluorescence may be used as a reporter of conformational changes at the discrete environment or in response to the discrete disruptive treatment.

In some embodiments, the persistence or the presence of a binding competent state can be measured by scattering signal originating from the immobilized molecule or tags attached to the immobilized molecule, for instance, a pair of nanoparticle tags.

In some embodiments, the immobilized molecule (e.g., aptamer, protein, or carbohydrate) may be modified to modulate the persistence of the binding competent state so as to increase or decrease the sensitivity of the biosensor. For example, proteins can be modified by a number of methods which can include mutation or crosslinking or forming disulfide bonds within the protein to maintain a selected conformation. In another embodiment, aptamers can be modified by adding or subtracting base pairs to the stem regions or by introducing single-nucleotide mutations. Without being bound by theory, the persistence of a binding competent state of an aptamer is dependent in part on the length or the total number of bases of the region(s) of internal self-complementarity or of total hydrogen bonding amongst different regions of the nucleic acid strands in the aptamer. A longer complementarity region or greater number of total bases, or a greater number of hydrogen bonds, or overlap between nucleic acid regions increases the persistence of the aptamer. Conversely, a shorter overlap or fewer bases involved in complementarity leads to a lower persistence or a lower stability of a particular conformation. Therefore, aptamers stability can be modified by mutation or adding stem regions.

### Persistence Measurements

A biophysical property of an immobilized molecule that changes upon analyte binding in a predictable, specific, and sufficiently large manner, and importantly, for all biosensor-analyte pairs so that analysis can be multiplexed, would dramatically simplify the design of a detection device.

The instant disclosure provides, inter alia, (1) methods for measuring the persistence or change in persistence of a surface-immobilized molecule having a binding competent state or ensemble of states when complexed with its binding partner and not complexed to its binding partner, upon a change in an environmental condition; and (2) systems and apparatus for measuring the same. Through practice of the disclosure herein, one may determine whether a surface immobilized molecule is complexed to its binding partner by assaying for persistence or changes in persistence of a particular conformation of the surface immobilized or otherwise bound molecule. This persistence may be assayed as a change in the stability of the surface immobilized molecule as a function of a change in an environmental condition. Alternately or in combination, persistence may be assayed for at a particular environmental condition (such as a particular temperature or particular voltage) under which a surface immobilized molecule is known to have differing states depending upon the presence or absence of a binding partner.

Without being bound by theory, the scientific principle of this disclosure is thought to be based on an immobilized molecule having a binding competent state or ensemble of states which can form a complex to a binding partner. Upon the application of an environmental perturbation (e.g., increased temperature or applied electric field), the persistence of the binding competent state of the immobilized molecule will be challenged. Upon the application of an extreme level of an environmental perturbation (e.g. a high applied electric field or a threshold level of heat), the higher order structure of the immobilized molecule will change. The persistence of the immobilized molecule in response to the environmental perturbation is different when bound to its binding partner compared to when the immobilized molecule is not bound to its binding partner In some embodiments, the binding partner is an analyte. In certain embodiments, this disclosure includes a biosensor for detecting an analyte based on the methods and systems described herein.

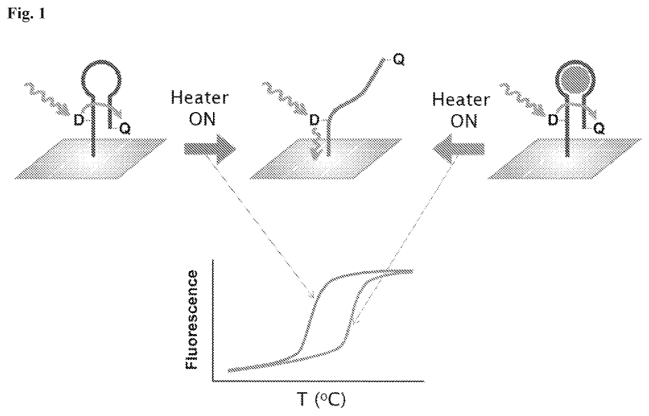

In some embodiments, the persistence of the immobilized molecule can be measured by indirectly measuring a conformational change of the immobilized molecule upon a change in environment, for example, an increase in temperature, as shown in FIG. 1.

In some embodiments, the immobilized molecule is a protein or an aptamer. Both proteins and aptamers represent promising starting points for sensor development because variants can be discovered that “fold” into a structure (or “state”) that specifically binds a target analyte (a binding competent state) and that may be subject to perturbation as a result of binding to a target such that assays for immobilized molecule folding state perturbation under a particular environmental condition or range of particular environmental conditions may indicate the presence or absence of binding to the target analyte. Moreover, analyte binding in some cases by definition stabilizes the binding competent state of the biosensor-if it did not, the analyte would not bind. Alternately, in some cases the analyte binding impacts the immobilized molecule structure so as to negatively impact the immobilized molecule's ability to emit a signal indicative of proximity of the fluorophores under a particular environmental condition or range of environmental conditions. That is, the analyte binding may stabilize the immobilized molecule or other detection molecule, but do so in such a way as to negatively impact its ability to fluoresce. Under suitable conditions, the binding competent state of any biopolymer may be induced to “unfold” and adopt a disordered and unstructured state or otherwise change its ability to emit a signal, and in the case of a biosensor, one that is no longer able to bind (or be stabilized by) the analyte. Thus, the presence of a target analyte will impact persistence, often by selectively stabilizing the binding competent state of the immobilized molecule. Consequently, the thermodynamic stability of the binding competent state of the immobilized molecule represents a biophysical property that will change in a specific and measurable manner, and importantly, for all sensor-analyte pairs.

Aptamers are oligonucleotides comprising DNA or RNA and that are able to assume a plurality of folded states which allow them to bind specific analytes with affinities and specificities that are sufficient to allow analyte detection, often via impacting aptamer structure. The strength of this binding may in some cases rival antibodies. Such folded states are referred to as “binding competent state(s).” However, unlike antibodies, aptamers can be rapidly developed against identified analytes and produced at a fraction of the cost of antibodies, including on large scale. Aptamers, like other structured oligonucleotides, undergo well defined and global transitions from their folded to their unfolded states (binding competent states to binding incompetent states). The unfolding transition has been measured while changing the temperature and determining the temperature at which half of the oligonucleotides remain in their binding competent state and half have binding incompetent state (for temperature as the environment, this temperature is referred to as the thermal melting midpoint, Tₘ). Change in persistence, often, represents a change in this thermal melting midpoint. The binding competent states are dominated by intramolecular interactions and are thus compact while the binding incompetent states are often (but not always) more extended in the vicinity of the reporter. Thus, most intramolecular distances between individual nucleotides often significantly increase upon aptamer unfolding, and decrease upon (re)folding. In addition, and unlike proteins, aptamers may be easily synthesized alone or with modifications, including dyes (including fluorescent dyes and quenchers or FRET pairs), introduced at any position. Therefore, pairs of dyes may be incorporated at positions that increase in distance from one another upon unfolding (and correspondingly decrease in distance from one another upon refolding). Because such pairs are known whose fluorescence is sensitive to the separation between the dyes (donor-quencher or FRET pairs, for example), they may be used to characterize the unfolding and refolding transitions (FIG. 1). While such donor-quencher and FRET pairs have been used to study the thermally induced (un) folding of structured oligonucleotides, their utility is based solely on their separation in the binding competent (e.g., folded) and binding incompetent (e.g., unfolded) states, and thus they should sensitively report on (un)folding induced by any means. Thus, fluorescence may serve as an indicator of donor-quencher or FRET pair proximity, such that if this proximity is positively or negatively impacted by target analyte binding, fluorescence will be informative as to this binding.

In some embodiments, an environmental perturbation is provided by an electric field Application of an exogenous electric field (E-field) has been commonly used to manipulate oligonucleotides in the form of purification methods (e.g., polyacrylamide gel electrophoresis (PAGE), capillary electrophoresis). In such oligonucleotide manipulations, the applied electric field exerts a force on the negatively charged backbone of the oligonucleotide, which causes it to migrate through the gel at a rate that depends on its electrophoretic mobility, a parameter which is dependent on the total number of charges and the hydrodynamic radius of the biomolecule. However, if the oligonucleotide is immobilized, translational motion is not possible, and instead the applied E-field, if strong enough, can induce a force on the oligonucleotide that stretches it out along the direction of the field lines (combing). Thus, if the immobilized oligonucleotide is initially folded, at a sufficient field strength, the force exerted by the E-field will cause it to unfold and adopt an extended, binding incompetent state. The field strength required to induce strand dissociation of duplex DNA (where one stand is immobilized) is determined by the stability of the duplex. The inventors have recognized that this process in some regards related to the unfolding of an aptamer, but it only measures duplex stability and cannot be used to detect an analyte. In analogy with thermally-induced unfolding characterized by Tₘ, the E-field-induced unfolding transition may be quantified by determining the voltage at which half of the structured oligonucleotides (duplexes or aptamers) unfold, which we refer to as Vₘ. With an aptamer, Vₘ will sensitively depend on the stability of the binding competent state, which in turn will depend on the presence or absence of an analyte that the aptamer binds.

Alternately or in combination, an environmental perturbation is effected through a change in temperature such as the addition of heat. Application of heat has commonly been applied to oligonucleotides to effect the disruption of intermolecular or intramolecular hydrogen bonding, among base pairs or otherwise responsible for the assumption of secondary structure. In such manipulations, as the heat applied to a structured oligonucleotide (or other binding agents such as antibodies or antibody fragments) reaches a threshold at which the structure of the oligonucleotide is lost (that is, as the temperature at which thermally-induced unfolding characterized by 1 m is reached). Binding to a target analyte may increase the persistence of the binding competent secondary structure. When the secondary structure impacts fluorophore-quencher or FRET pair proximity, fluorescence may be used to assay for secondary structure configuration at a given temperature or across a range of temperatures, which may be indicative of target analyte binding.

Yet other forms of environmental perturbation are consistent with various embodiments of the disclosure herein. Physical perturbation, as effected through vibration or sonication, may also effect an environmental perturbation sufficient to visualize a perturbation in binding state. Similarly, changes in local conditions such as ionic concentration, buffer composition, local hydrophilicity or hydrophobicity are also consistent with the disclosure herein, as are other local condition changes that may effect an environmental perturbation sufficient to visualize a perturbation in binding state persistence indicative of target analyte binding.

Affinity reagent such as aptamers or antibodies that specifically bind target analytes of interest such as specific proteins, hormones, lipids, viral particles or types of cells can be used. Beneficially, aptamers can be developed using any of a number of established SELEX methods (Systematic Evolution of Ligands by EXponential Enrichment) (Tuerk et al., Systematic evolution of ligands by exponential enrichment: RNA ligands to bacteriophage T4 DNA polymerase. Science (New York, NY). 1990; 249(4968):505-10; Ellington et al., In vitro selection of RNA molecules that bind specific ligands. Nature. 1990; 346(6287):818-22; Robertson et al., Selection in vitro of an RNA enzyme that specifically cleaves single-stranded DNA. Nature. 1990; 344(6265):467-8; Lee et al., Aptamer therapeutics advance. Curr Opin Chem Biol. 2006; 10(3):282-9; Banerjee et al., Aptamers: multifunctional molecules for biomedical research. J Mol Med (Berl). 2013; 91(12):1333-42; Hong et al., Single-stranded DNA aptamers against pathogens and toxins: identification and biosensing applications. Biomed Res Int. 2015; 2015:419318; Zhang et al., Practical application of aptamer-based biosensors in detection of low molecular weight pollutants in water sources. Molecules (Basel, Switzerland), 2018; 23(2):344). Third-party synthesized or designed aptamers can also be purchased commercially. Once discovered, aptamers can be synthesized by solid phase synthesis with modifiers such that dye pairs can be included in the aptamer sequence or connected to a modified nucleotide at specific locations that result in different emission properties in the binding competent and binding incompetent states. Such modifiers can be commercially obtained (e.g., IDT DNA, LGC Biosearch Technologies, or Trilink, Inc. (a subsidiary of Miravai Life Sciences (California) at time of filing).

In some embodiments, a plurality of biomolecules such as affinity reagents can be immobilized to a region of a substrate to form a “cluster.” Often, the affinity reagents of a cluster have the same identity or share a common binding moiety. However, in some alternatives heterogeneous cluster, or clusters having more than one population of binding moiety, are also contemplated and consistent with the disclosure herein.

Substrates are generally locally flat. Some substrates are flat surfaces, such as those of a chip or a region of a flow cell. Alternately, some substrates are locally flat beads, such as spherical beads. Many of these beads comprise one cluster per bead, the cluster being in some cases homogeneous or heterogeneous. Beads may be deposited in wells, such as wells that are configured to accommodate no more than one bead per well. Beads in these cases are configured to be amenable to sequencing reactions, such that a bead in a well may have a nucleic acid of its cluster sequenced so as to identify the binding moiety of a cluster on a bead at a position on a well array. The sequencing is in some cases of a barcode or other distinct tag identifier. Alternately, particularly in the case of aptamers, the aptamers of a cluster are sequenced directly for a bead in a well so that the signal from that well may be correlated to detection of a particular target analyte. Alternatively, the identity of each bead on the array can be determined by a decoding process. In one embodiment, the decoding process identifies the different bead types by using sequential hybridization of pools of fluorescently-labeled complementary decoder probe sequences (Gunderson et al. 2004; Vickovic et al. 2019). The decoder probes are stripped from the bead array between decoder pool hybridization steps.

Alternately, in some cases affinity reagents such as aptamers or antibodies are deposited into an aqueous volume such as a well or an emulsion droplet without being bound to a solid surface.

The system can comprise a CMOS sensor or CCD. Fluorescence from each cluster may then be monitored by the CMOS detection layers in the presence of an E-field, a thermal source (e.g. joule heating) or other environmental perturbation acting at the substrate surface. The magnitude of the E-field, temperature or other environmental perturbation can be varied from zero to a strength sufficient to unfold all aptamer types, and by measuring fluorescence as one embodiment of the aptamer persistence, the voltage, temperature or other environmental condition at which each individual cluster of aptamers transition from their folded to binding incompetent state (with an increasing E-field strength, temperature or other environmental perturbation) or from their binding incompetent to binding competent state (with a decreasing E-field strength, temperature or other environmental perturbation) can be determined, both in the presence and absence of a biological solution whose analyte composition is of interest. Alternately, when the level of persistence for a target analyte or set of target analytes is known, a single level or a set of levels of environmental perturbations, such as a set temperature, may be selected such that bound and unbound aptamers or other binding moieties may be distinguished. If the biological solution contains the analyte recognized by a given aptamer, its binding will stabilize the folded, binding competent state or ensemble of states of that aptamer and result in an observable shift of its unfolding voltage to a higher value and its refolding voltage to a lower value.

Analyte-bound surfaces are subjected to a range or gradient of environmental condition change, such that as the Tₘ, Vₘ or other threshold parameter for a given environmental condition is met or passed, or when the level of persistence is surpassed for a given analyte-binding moiety pair, a corresponding change in fluorescence is observed. When the threshold parameter is consistent with persistence caused by target analyte binding, the sample may be scored as having the target analyte or analytes.

Alternately, in some cases a single environmental condition value is selected, such as a single voltage or a single temperature, which is known to fall outside of the stability range for an unbound binding moiety such as an aptamer, but to fall within the persistence stability range for that binding moiety bound to a target analyte. The surface is subjected to that temperature (or other environmental perturbation) and binding moiety fluorescence is assayed. This approach May be used for a single temperature or other environmental perturbation, or may comprise a plurality of temperatures or other environmental perturbations selected to visualize secondary structure persistence for a plurality of binding moieties having various different Tₘ, Vₘ or other threshold parameters. In some cases a single environmental condition value is selected, and a plurality of samples are run across or contacted to the surface successively such as over time, so as to effect iterative sampling or assaying of samples such as temporally distinctly collected or provided samples.

Consistent with single environmental perturbation assays, in some cases the binding moieties for a given surface are selected so as to have Tₘ, Vₘ or other threshold parameters, or persistent shifts that all fall within a common range, such that a single temperature or other environmental shift is sufficient to visualize the target analyte-sensitive fluorescence status for a substantial proportion or all of the binding moiety clusters on a surface. That is, in some cases a single application of a temperature is sufficient to assay for target analyte binding status for a plurality of clusters, or such that a surface may be assayed through a single temperature or other environmental perturbation, rather than subjecting a surface to a temperature or other environmental perturbation gradient so as to span a plurality of Tₘ, Vₘ, or other threshold parameter thresholds or to fall within a plurality of persistence ranges.

The size of each cluster can range from about 30 microns in diameter, and have a circular or semicircular shape. In some embodiments, there can be up to 2000 aptamer clusters arrayed on a 2 mm×2 mm CMOS sensor substrate, which will allow for a point-of-care device that simultaneously, inexpensively, and rapidly detects ˜60 analytes of choice present in different 16 biological solutions. However, a broad range of shapes and cluster sizes are consistent with the disclosure herein. In some cases cluster size is limited by the optical capacity of the detection device, such that cluster sizes are limited by the pixel size of the detection device. Alternately, larger clusters are also consistent with some embodiments of the technology, such as 50, 100, 200, 300, 400, 500 or greater than 500 microns. Some surfaces are ‘coated’ with constituents of a single cluster, such that all or a substantial portion of the surface comprises a single binding moiety lawn rather than distinct clusters.

### Methods—Assay Format

A representative embodiment to perform the methods described herein is depicted in FIG. 1, which is based on the site-specific incorporation of two dyes into the aptamers. The first dye (the donor dye (D)), is continuously excited by an LED source. In the “D-Q” assay format (FIG. 1), the second dye is a quencher (Q) that is positioned within the aptamer such that in the aptamer's binding competent state it efficiently quenches D, but not in the more extended, binding incompetent (unfolded) state. In another embodiment (the “D-A” assay format), the second dye is an acceptor (A) that efficiently undergoes FRET with D in the binding competent state, but not in the more extended binding incompetent state. Thus, in the D-Q format, the unfolding transition is revealed by the appearance of fluorescence from D, while in the D-A format, it is revealed by the appearance of fluorescence from D and a concomitant disappearance of fluorescence from A.

As described herein, in some embodiments each aptamer cluster will be subject to a varying environmental condition such as temperature or electric field, starting at a lower temperature or voltage value that does not destabilize unbound binding moieties such as aptamers, and changed to a value at which the aptamers of some or all clusters unfold at a rate at which individual cluster destabilization may be temporally detected, and then optionally back to original value or to a level at which cluster binding moieties such as aptamers are stable. The (un) folding-induced changes in the fluorescence of the dyes (D or A) of each aptamer cluster will be detected by the CMOS sensor photodiodes or other sensors, both in the presence and absence of the biological solution of interest. As explained in more detail herein, the presence of a target analyte will be revealed by an observed shift in the values of the environmental condition such as temperature or field strength required to induce (un) unfolding of the corresponding aptamer (e.g. a shift in Tₘ or Vₘ).

The specific steps of performing some representative embodiments are further described. The fluorescent aptamer-modified CMOS sensors are optionally first to be conditioned by washing with a buffer. Under constant LED irradiation to excite dye D, the aptamers will then be subject to a temperature increase or an electric field swept from 0 or other value for which the binding moieties remain stable to a sufficiently high value to unfold all aptamers. Initially, in the D-Q format no fluorescence will be observed as all aptamers will be in their binding competent states and thus the continually excited D will be efficiently quenched. Initially, in the D-A format, maximal fluorescence from A will be detected. In both cases, as the energy transferred to the aptamers is increased by applying heat or a repulsive coulomb force (e.g. E-field applied via a voltage source), aptamers will begin to unfold, challenging the persistence of the binding competent state, and fluorescence from D will appear and increase in intensity (and in the D-A formant, fluorescence from A will simultaneously disappear). The temperature or voltage at which 50% of the unbound aptamer unfolds (Tₘ^{ub} or Vₘ^{ub}) will be determined. The temperature or voltage will be set back to zero or to a stabilizing level and the chip will be prepared for sample analysis.

Biological samples may first be filtered or otherwise pre-treated, or applied directly or ‘raw’ as drawn from a patient and incubated with the aptamer functionalized CMOS chip and optionally washed with buffer solution to remove unbound components. Chips will comprise clusters of high target affinity reagents such as aptamers (in addition, different aptamers can be used to create different clusters that recognize the same target to improve reliability of detection) and optionally clusters of lower differing affinity reagents such as aptamers. While sweeping increasing the unfolding force environmental condition such as by increasing temperature or by applying voltage gradient (e.g. electric field), the temperature, condition or voltage magnitude at which 50% of the aptamers at each high affinity cluster unfolds (the observed Tₘ, Tₘ^{obs}, Vₘ, Vₘ, Vₘ^{obs}) will be determined as described above. Because analyte binding, by definition, will selectively increase the stability of the bound aptamer, under many aptamer configurations persistence will be observed such that a higher temperature or a stronger repulsive coulomb force via E-field will be required to induce the unfolding transition, relative to clusters that do not exhibit target analyte binding. Thus, aptamer clusters whose analyte is present will unfold at a higher temperature, Tₘ^{obs}=Tₘ^{b}>Tₘ^{ub}, or a higher observed voltage, Vₘ^{obs}=|Vₘ^{b}|>|Vₘ^{ub}|, where |Vₘ^{b}| is the voltage magnitude at which 50% of the bound aptamer is unfolded. Thus, comparison of Tₘ^{obs} or Vₘ^{obs} to the saved previously measured Tₘ^{ub} or Vₘ^{ub} (that is, observing fluorescence measuring change in persistence by fluorescence) will allow for the determination of which analytes are present in the biological solution (FIG. 1).

When an analyte is detected via a fluorescence persistence shift such as Tₘ^{obs} or Vₘ^{obs} values measured at high affinity clusters, the fluorescence at variable affinity clusters corresponding to the analyte will be examined. If the analyte is authentically present, the variable affinity clusters will show confirmatory fluorescence behavior, and in fact, behavior that allows for the concentration of the analyte to be determined. Specifically, with a sufficiently decreased affinity or a sufficiently low concentration of target analyte, not all aptamers of a given cluster will be bound, and thus when the temperature or the voltage is swept, these clusters will show a detectable biphasic threshold for change in fluorescence (in the case of voltage sweeps, both Vₘ^{b} and Vₘ^{ub} will contribute to the observed transition). With consultation of chip-standardized calibration curves, this will allow for the concentration of the analyte to be determined. In this manner, with a properly designed set of aptamer sensors, including multiple aptamers that bind analytes with high affinity and several that bind with reduced affinities that span the range of analyte's physiological concentrations, or clusters that exhibit multiple binding moiety densities, or both, the chip will enable accurate multiplex detection of analytes and the quantification of their concentrations in a single experiment.

A number of approaches for quantifying target analyte amount or concentration in a sample are contemplated herein. As mentioned above, some approaches use multiple clusters which bind a common target analyte with different affinities, such that distinguishing among clusters which exhibit binding to the target analyte may indicate target analyte concentration. That is, applying a sample to a surface having a first cluster that binds the target analyte at a first affinity and a second cluster that binds the analyte with an second affinity, and assaying for signal from each of the first cluster and the second cluster, may indicate that the target analyte is present at a concentration sufficient to exhibit binding ay both the first and the second cluster, at only one of the first or second cluster, or neither the first nor the second cluster. In various embodiments one may also employ a third cluster that binds the target analyte at a third affinity, or higher numbers of varying affinity clusters.

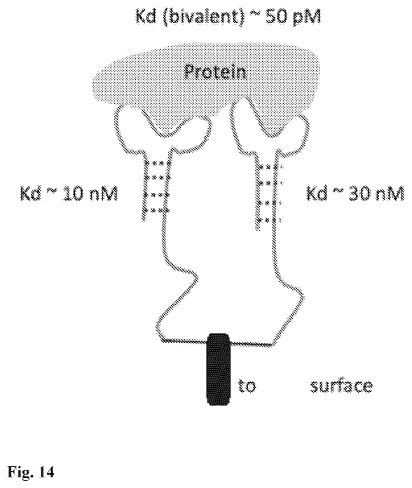

Exemplary surface clusters for quantification comprise a first cluster population having a first binding moiety that binds with a first affinity; a second cluster population having a second binding moiety that binds to the target molecule at a second region and optionally at a second affinity, and a third cluster population that comprises both first binding moieties and second binding moieties, so as to bind the target molecule with a third, higher effective affinity. The third cluster may be heterogeneous in that both first binding moiety molecules and second binding moiety molecules are bound at the third cluster. Alternately, the first binding moiety and the second binding moiety may be joined, for example by a common phosphodiester backbone, to form a chimeric binding molecule, which is deposited in a homogeneous cluster that nonetheless comprises a heterogeneous binding moiety population. See FIG. 14A.

Cluster binding affinity may be modulated through a number of approaches. For example clusters may comprise independently derived binding moieties, such as aptamers that were independently generated from separate SELEX reactions, and that exhibit separate binding affinities to a common target analyte. Alternately, a common binding moiety such as a common aptamer may be modified to produce a derivative aptamer, so as to impact but not abolish target analyte affinity. Clusters of both the unmodified and the modified aptamer may be used to form a first cluster and a second cluster, respectively, exhibiting separate binding affinities to the common target. Yet another option comprises varying the tethering chemistry or moiety that tethers a common bonding moiety to form a first cluster and a second cluster, such that, for example, aptamers at a first cluster are tethered more closely to the surface than are aptamers at a second cluster, resulting in the clusters differing in their binding affinity to the target analyte. Yet another option comprises varying the concentration of aptamers among various clusters, such that the clusters may exhibit different binding affinities to the target analyte. Aptamer concentration May be reduced such that the absolute cluster aptamer concentration is reduced, or alternately some clusters may be diluted by the presence of nonbinding aptamers so as to form heterogeneous populations that differ in binding affinity as the concentration of target analyte-binding aptamers decreases. Yet another option comprises arraying multiple clusters having either identical or differing binding affinities for the common analyte, serially diluting the sample, and quantifying the target analyte in the sample by counting the number of clusters which bind to the target analyte in the diluted sample.

### Methods—Measuring Cluster Signals

A number of measuring approaches are consistent with the disclosure herein. In some embodiments, measuring can be performed using single-scan detection, where each of a plurality of immobilized molecules comprising a dye or dye pair is detected in one step. In some embodiments, the folding/unfolding process can be repeated on the same sample.

In some embodiments, a sample is first contacted to an immobilized molecule. In some embodiments, the immobilized molecule with the sample (which can comprise a binding partner) can optionally be subject to a wash step. In some embodiments, the wash step is performed directly before, or directly after presentation of the binding partner after measuring the persistence of the binding competent state of the immobilized molecule.

Some embodiments of the disclosed technique involve contacting an immobilized molecule with particular types of sample, wherein the sample comprises a binding partner. In some embodiments, the sample does not comprise a binding partner. The assay will determine whether the binding partner is present in the sample. According to one embodiment, persistence is measured during a change in the environment (e.g., introducing a new chemical buffer composition, or changing the electric field). In some embodiments, signal indicating the persistence is measured for a time ranging from 1 nanosecond to 10 minutes, from 1 nanosecond to 1 minute, from 100 nanoseconds to 1 second, from 1 millisecond to 500 milliseconds, or between any of the aforementioned times.

Alternately, persistence is in some cases measured by assaying the surface at a single temperature that falls within the persistence change in stability which occurs upon binding to a target analyte. Rather than ramping up an environmental change such as temperature, the surface is heated rapidly to a temperature that divides bound, stabilized cluster fluorescence from unbound fluorescence levels. Surfaces may be also assayed at a temperature at which all clusters are bound or in a stabilized state, and also may be assayed at a temperature at which all clusters are in a denatured or unbound state. However, in these embodiments, heating or other environmental changes are rapid and targeted, and measurements are made at particular, targeted states of environmental change such as particular temperatures, rather than measuring changes in response to ongoing, continuous change in an environmental condition such as temperature. Accordingly, these persistence measurements are executed more quickly than are ongoing gradual change measurements. In some embodiments, these targeted state measurements are performed on surfaces where a plurality, a majority or substantially all of the clusters exhibit persistence shifts across a common temperature range, for example, such that a continuous temperature change measurement is not needed to detect binding states across the clusters on a surface.

Nonfluorescent detection methods are also consistent with the disclosure herein. For example, radiometric electrochemical biosensors are consistent with the disclosure herein, such as Wang et al. (2022) “An ultrafast ratiometric electrochemical biosensor based on potential-assisted hybridization for nucleic acids detection” Analytica Chima Acta Vol 1211 Jun. 8, 2022, which is hereby incorporated by reference in its entirety. Some such methods comprise, for example, labeling an immobilized detection reagent with an electrochemical detection reagent such as ferrocene, and converting redox ions at the surface near a detection event to an electronic signal. Alternative nonfluorescent detection approaches are consistent with the disclosure herein, such that not all approaches are limited to fluorescent detection. In particular, radiometric and electrochemical signals are consistent with the disclosure herein.

### Affinity Binding Reagents

A number of affinity binding reagents are compatible with the disclosure herein. Exemplary affinity binding reagents include antibodies, antibody fragments such as Fab regions or Fv regions, and aptamers, but may also include a broad range of moieties to which a target analyte may bind, such as oxygen and carbon monoxide binding by heme or hemoglobin complexes. Substrates, protein complex partners, cell surface proteins, ligands, receptors, epitope harboring molecules, or any number of target analyte binding partners are consistent with the disclosure herein. In some preferred embodiments, aptamers are affinity bind reagents, and are used to identify non-nucleic acid target analytes.

In some embodiments, the affinity binding reagent biomolecules of this disclosure are aptamers. Aptamers can be commercially sourced or developed against a specific target (which will become the analyte). The aptamers can be developed using the SELEX method (Systematic Evolution of Ligands by Exponential Enrichment) (Tuerk et al., Systematic evolution of ligands by exponential enrichment: RNA ligands to bacteriophage T4 DNA polymerase. Science (New York, NY). 1990; 249(4968):505-10; Ellington et al., In vitro selection of RNA molecules that bind specific ligands. Nature. 1990; 346(6287):818-22; Robertson et al., Selection in vitro of an RNA enzyme that specifically cleaves single-stranded DNA. Nature. 1990; 344(6265):467-8; Lee et al., Aptamer therapeutics advance. Curr Opin Chem Biol. 2006; 10(3):282-9; Banerjee et al., Aptamers: multifunctional molecules for biomedical research. J Mol Med (Berl). 2013; 91(12):1333-42; Hong et al., Single-stranded DNA aptamers against pathogens and toxins: identification and biosensing applications. Biomed Res Int. 2015; 2015:419318; Zhang et al., Practical application of aptamer-based biosensors in detection of low molecular weight pollutants in water sources. Molecules (Basel, Switzerland), 2018; 23(2):344) and/or made by solid phase synthesis from external suppliers (e.g. IDT DNA).

The SELEX process is initiated with a pool of oligonucleotides referred to as a “library.” Starting libraries can include a central stretch of 20-40 randomized nucleotides and two flanking regions of fixed sequence for primer-binding during PCR amplification. However, more complex library architectures can be designed to bias the aptamer sensor towards a desired secondary structural motif. For example, three constant regions may be present including two terminal primer-binding sites and a centrally positioned DNA hairpin separating two randomized regions of diversity. Typical libraries are comprised of 10\hat{ }14-10\hat{ }15 oligonucleotide members. Sometimes, the target analyte of interest will be immobilized on agarose beads or another solid support and incubated with the oligonucleotide library. After incubation, the solid support will be washed to remove unbound library members, and then with denaturing solution to release the bound oligonucleotides. The stringency of these positive rounds of selection can be increased by a combination of decreasing target concentration and increasing the number of wash steps. Rounds of negative selection can also be included, initially via incubation with beads without the target analyte, in which case the unbound fraction of the library is retained and the bound fraction is discarded. During later rounds, pressure for selective binding can be increased by performing the selections in the biological media of interest (i.e. blood). After each round, the recovered oligonucleotides can be PCR amplified, and either subjected to additional rounds of selection or sequenced to determine member identity. Candidate aptamers can then be individually prepared and analyzed. Aptamer affinity for the target analyte can be determined using a range of standard methods, such as surface plasmon resonance (SPR), in particular the Biacore T200 system (Cytiva/Danaher).

In some embodiments, the aptamers can comprise a nucleotide as described herein. In some embodiments, the nucleotide can be further functionalized, either before SELEX, during SELEX, or after SELEX, and the functionalization be as described herein. In some embodiments, the aptamer nucleotide functionalization can comprise conjugation to one or a plurality of dyes or dye pairs.

The selected aptamers can be synthesized with D (donor) and Q (quencher) or A (acceptor) dye pairs and their fluorescence characterized in both their folded and chaotropic or heat-denatured states. The dye-labeled aptamers with the desired fluorescence properties (FIG. 1) can be selected and immobilized to a modified CMOS sensor (see below) and subjected to a temperature sweep or a voltage sweep, and their force-induced fluorescence behavior confirmed. In some embodiments, the aptamer can comprise a 3′- or 5′-modifier which presents a conjugation site to a surface.

Some affinity binding reagents comprise a single binding moiety. Alternately, some affinity binding reagents comprise two or more than two species of binding moieties, for example targeting different regions of a target analyte. Such affinity binding reagents are often joined by a common covalent linkage such as a common phosphodiester backbone. That is, two fused aptamers that target separate regions of a common target analyte may form a single affinity binding reagent. Alternately, in some cases a cluster may comprise a heterologous population of two or more affinity binding reagents, that in some cases target a single target analyte, for example at two positions on the analyte.

Also disclosed herein are populations of affinity reagents such as aptamers that share a common environmental condition and parameter at which they exhibit a change in a reporter indicative of target analyte binding. Such populations may comprise diverse affinity reagents such as aptamers that separately bind biochemically diverse target analytes under a common set of reaction conditions. In some cases the populations differ by no more than 5, 4, 3, 2, 1, 0.5 or less than 0.5 degrees Celsius in the temperature at which their reporter activity changes when bound to their diverse target analytes. In some cases the populations comprise at least 10, 20, 50, 100, 200, 500, 1,000, 2,000, 5,000, 10,000 or more than 10,000 distinct affinity binding reagents. In some cases these populations consist of aptamers, or another uniform category of affinity reagent such as antibodies. Alternately, some populations comprise a diversity of affinity reagent categories, such as aptamers, antibodies, antibody fragments, ligands or other binders. Aptamers are generally most readily synthesized, and easiest to tailor to a specific temperature at which their reporter activity changes. Accordingly, in some cases a particular preferred antibody or other affinity reagent is selected, and a diverse aptamer population is synthesized having a common temperature at which their reporter activity changes so as to be compatible with the non-aptamer affinity reagents of the population.

In some cases, affinity reagent populations are selected for a common biochemical pathway or set of biochemical pathways, a common disease or set of diseases to be detected, or other common indication or indications. These populations are in some cases also selected to have a common temperature at which their reporter activity changes, so as to facilitate rapid analysis.

Some affinity reagents are selected for breadth of target analyte rather than rapidity of assay execution, or both breadth and rapidity of execution. Examples of these affinity reagent populations are those that bind, for example, each member of a single viral proteome or a category of viral proteomes, such as coronaviruses, retroviruses or other viral category, cellular pathogens, endogenous disease markers, cancer markers or other broad categories of targets. More broadly, some affinity reagent populations are selected to assay for, for example, some or all known bloodborne disorders, some or all known cancer markers, some or all known tropical diseases, some or all known apicomplexan diseases, some or all known trypanosome diseases, some or all known yeast or fungal diseases, some or all known sexually transmitted diseases, some or all known autoimmune disorders, some or all known serotonin or endorphin-related psychological disorders, or multiple combinations of members of these or other diagnostic categories of interest. Accordingly, disclosed herein are affinity reagent populations that assay for 1, 2, 3, 4, 5, 6, 7, 8, 9, 10, 20, 50, 100, 200, 500, or more than 500 target analyte markers of 1, 2, 3, 4, 5, 6, 7, 8, 9, 10, 20, 50, 100, 200, 500, 1,000, or more than 1,000 disorders, diseases, indications or conditions. In many embodiments, the target analytes comprise at least some non-nucleic acid targets, and often a majority of non-nucleic acid targets. Often, target analyte detection does not comprise base pairing of target analytes to aptamers.

The populations of affinity reagents above are in some cases deployed as clusters on arrays, either by direct binding to effect immobilization onto the arrays, or by binding of copies of individual target analyte binders onto beads that are deployed into wells, or by direct delivery of copies of individual target analyte binders into constrained volumes such as wells or droplets, for example emulsion droplets. Accordingly, the populations described herein are readily deployed on arrays, such as the arrays below, on systems comprising these arrays, and employed in the practice of methods disclosed throughout the present disclosure.

### Arrays

This disclosure provides for biosensors, in particular biosensors for detecting the presence of an analyte in a sample according to the methods described herein. The biosensor can comprise a plurality of immobilized molecule clusters (e.g., immobilized biomolecule) configured on a surface as an array. The array is an affinity reagent array, such as an antibody array or an aptamer array. In some embodiments, the array can be made by contacting aptamers functionalized as described above with a functionalized surface using an “inkjet” like technology, wherein a microdroplet (or smaller volume than a microliter) is deposited at a selected location on the substrate. In some embodiments, the functionalized substrate can be prepared by treating with oxygen plasma followed by water for surface activation and silanol repopulation. In some embodiments, the functionalized substrate can be prepared without water treatment. The functionalized substrate can be prepared using a commercial CVD/plasma system (e.g., EasyTube 100, FirstNano (NY)) The functionalized substrate can be functionalized with an appropriate bioconjugation target appropriate for the bioconjugation site partner on the functionalized aptamer. For example, after aptamer sequence identification, the oligonucleotide sequence can be commercially sourced (e.g., IDT DNA) with a 5′-amino modifier. The surface can be functionalized with a NHS (N-hydroxy succinimide) functionalized silane (Gelest). The aptamer can be contacted with the NHS-functionalized surface to react 5′-amino modified aptamer to the NHS-moiety on the surface to covalently bond the aptamer to the surface. Numerous methods exist to conjugate oligonucleotides to surfaces which can include or exclude thiol/maleimide, click chemistry (azide/alkynyl, e.g., dibenzocyclooctyne (DBCO)), carboxylic acid/amine, carboxylic acid/alcohol, amine/halide, etc., using appropriate bioconjugation pairs.

In some embodiments, the immobilized molecules, or clusters of the same immobilized molecule identity, can be isolated from immobilized molecules of a different identity, and all immobilized molecules subject to an environmental perturbation.

After deposition of the aptamer solution onto the functionalized substrate, the aptamer cluster can be round and have a diameter from 1 to 500 microns, based on the concentration, spotting solution composition, temperature, humidity, and surface density of 8 functional molecules. In some embodiments, the aptamer cluster is about 30 microns in diameter with a pitch of about 45 microns. The pitch can range between 10 microns to several millimeters. The pitch can be controlled by the selective deposition locations. The purpose of decreasing the pitch is to increase the number of aptamer clusters per unit area. However, the pitch must not be too short to reduce the likelihood of crossover from one aptamer cluster to a neighboring aptamer cluster. With the pitch at 45 microns and the cluster diameter at 30 microns, the resolution (or density per unit area) of the aptamer clusters will be about 600 dpi. In some embodiments, clusters can be formed on selectively functionalized substrates, so as to enforce an ordered array of aptamer clusters. Aptamer clusters will therefore be comprised of tens of millions of individual aptamers and their fluorescent signal will be easily resolved above detection limit with modified commercial CMOS sensors. Each aptamer cluster can be printed in replicate (e.g., from 2 to 100 replicates or more, preferably 3 to 50, and more preferably 4) to ensure at least three independent measurements for each aptamer type in case one of the clusters fails to perform during the test or manufacturing process, and/or to obtain statistical reproducibility and error measurement associated with the assay. With a typical 45 micron pitch, about 2,000 clusters can be fit on a 2 mm×2 mm substrate. Such a configuration affords the simultaneous detection of about 60 different analytes in a biological sample which should be more than sufficient for most clinical applications because typical blood test panels analyze between 5 and 30 analytes.

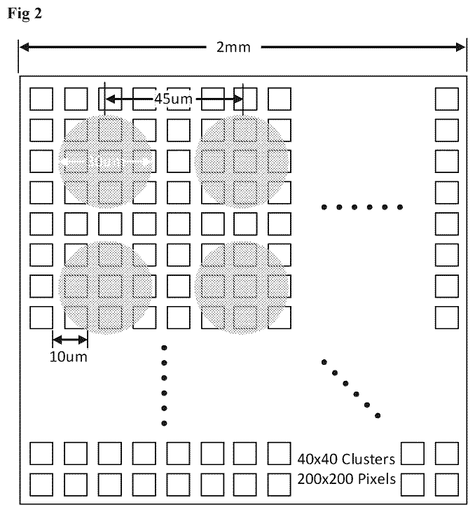

The configuration of one representative embodiment depicting the biomolecule cluster overlayed with the imaging sensor elements on a substrate is depicted in FIG. 2.

Alternately, arrays are in some cases formed through the deposition of cluster-coated beads onto a surface, such as into wells on a surface. The wells are configured to accommodate no more than one bead at a time, such that well positions effectively act as cluster positions in these systems. To determine the identity of the bead cluster deposited within a particular well, a signal identifying the bead is generated. Such a signal may comprise sequencing a tag or label tethered to the beads, or may comprise sequencing aptamers of an aptamer cluster of the bead directly, thereby identifying the bead and the affinity reagent responsible for the analyte signal at that position or that well in a well array. Alternatively, the identity of each bead on the array can be determined by a decoding process. In one embodiment, the decoding process identifies the different bead types by using sequential hybridization of pools of fluorescently-labeled complementary decoder probe sequences (Gunderson et al. 2004; Vickovic et al. 2019). The decoder probes are stripped from the bead array between decoder pool hybridization steps

In yet further alternatives, affinity reagents are deposited unbound into isolated volumes, such as emulsions or wells of an array. Aliquots of a sample are deposited into the isolated volumes, and binding assays are performed as disclosed herein. The affinity binding reagent is in some cases known prior to deposition at a particular isolated volume such as a well position. Alternately, in some cases an affinity reagent is isolated subsequent to target analyte assaying, and is then its identity is determined through sequencing of the affinity reagent aptamer directly, or of a tag co-deposited with the affinity reagent in the isolated volume.

The arrays disclosed herein are compatible with harboring affinity reagent populations such as those disclosed above, and for use in the systems and methods disclosed throughout the present disclosure.

### Flow Cell

In some embodiments, the contacting step is facilitated by the use of a reaction vessel. A “reaction vessel” is a substrate to which a molecule can be immobilized or otherwise localized. The reaction vessel allows for the presentation of an applied environmental perturbation to the immobilized molecule in (or on) the reaction vessel. In some embodiments, the reaction vessel is a flow cell or chamber, multiwell plate, bead, etc. Flowing liquid reagents (which can include or exclude the sample or a wash solution) through the flow cell, which contains an interior solid support surface (e.g., a surface) conveniently permits reagent exchange or replacement. Immobilized to the interior surface of the flow cell is one or more immobilized molecules using the methods described herein. In some embodiments, flow cells are in fluidic communication with microfluidic valving that permits delivery of liquid reagents (e.g., components of the “reaction mixtures” discussed herein) to an entry port. Liquid reagents can be removed from the flow cell by exiting through an exit port. Optionally, liquid reagents can be moved back and forth within the flow cell, for example, to effect mixing.

### CMOS Substrate

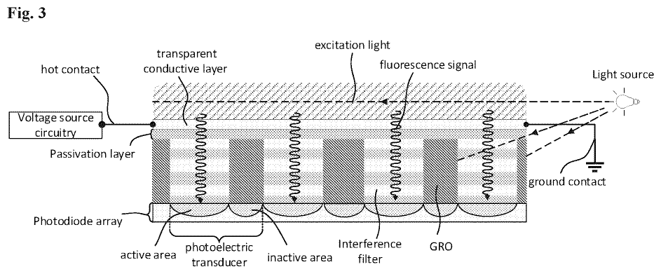

In some embodiments, a biosensor such as the biosensor in FIG. 3 comprises a biosensor comprising. (a) an excitation light source, (b) an array of detection elements comprising a plurality of sets of stacked layers, comprising: (i) a first set of stacked layers comprising a top layer which is a transparent conductive layer configured to support an immobilized molecules; and (ii) a second set of stacked layers comprising an optical filter (e.g. interference filter) and a solid-state photodiode array; wherein the excitation light source can be coherent or incoherent, wherein the light excitation source may include an optical collimator, wherein the photodiode array comprises an array of a photoelectric transducer unit which converts the photons of received light to electrons, wherein the photoelectric transducer unit includes an active area and an inactive area, wherein the number of electrons generated by a single photoelectric transducer is proportional to the number of received photons in the active area, wherein the inactive area includes electronic and digital circuitry needed to operate the photoelectric transducer unit, wherein the transparent conductive layer is acting as a resistive heater and the voltage source circuitry apply controlled voltage across the transparent conductive electrode, with the hot contact and the ground contact as depicted in FIG. 3, wherein the optical filter is operably coupled to the transparent conductive layer and the solid-state photodiode array, wherein a passivation layer (e.g. SiO₂) may be present between the transparent conductive layer and the optical filter, wherein the immobilized molecules comprises a nucleic acid sequence, a dye pair, and one or a plurality of binding competent state(s), wherein the optical filter comprises a plurality of vertical alternating dielectric layers (e.g. interference filter) which are laterally separated by a dielectric grid with an optically opaque surface (GRO), wherein a unit cell of the GRO defines the lateral boundaries of a detection element (i.e. pixel), wherein the GRO is laid over the inactive of the photoelectric transducer array, wherein the GRO comprises a stack of dielectric and metal oxide layers in which conductive routing layers may be embedded for electric connectivity, wherein the height and the pitch of the GRO define the field of view of the photodiode array (FOV), wherein the dye pair is configured to be positioned within the FOV and the excitation light source is configured to be exterior to the FOV, and wherein the filter layers are configured to transmit the emission signal from the dye pair when the dye pair is subject to a light source from the excitation light source, and to reflect the background excitation light that is not blocked by the walls. The operable coupling between the optical filter and the transparent conductive layer can be a physical connection. In some embodiments, the dye pair of the biosensor can be covalently linked to the nucleic acid sequence. In some embodiments, the solid-state photodiode array can be fabricated using CMOS technology. In some embodiments, the solid-state photodiode array can be configured to detect the emission signal from the dye pair when the dye pair is subject to a light source from the excitation light source.

In some embodiments, the biosensor comprises a detector surface that can be functionalized (e.g. chemically or physically modified in a suitable manner for attaching an immobilized molecule). For example, the detector surface can be functionalized and can include a plurality of reaction sites having one or more biomolecules immobilized thereto. The detector surface can have a reaction array of reaction recesses. Each of the reaction recesses can include one or more of the reaction sites. The reaction recesses can be defined by, for example, an indent or change in depth along the detector surface. In other examples, the detector surface can be planar.

In some embodiments, the biosensor comprises a CMOS photodetector array. The CMOS photodetector array comprises a sensor array as described herein. In some embodiments, the CMOS photodetector array can include a plurality of stacked conductive routing layers (e.g. conductors, traces, vias, interconnects, etc.) that are capable of conducting electrical current, such as the transmission of data signals that are based on detected photons. A photodetector array comprises an integrated circuit having a planar array of the light sensors (i.e. photoelectric transducers). The circuitry formed within detector can be configured for at least one of read out signals from light sensors after an exposure period (integration period) in which charge accumulates on light sensor, signal amplification, digitization, storage, and processing. The circuitry can collect and analyze the detected emissions signal light and generate data signals for communicating detection data to a bioassay system. The circuitry can also perform additional analog and/or digital signal processing in detector. Light sensors can be electrically coupled to circuitry through gates.

In some embodiments, the solid-state photodetector array can comprise a detector which can be provided by a solid-state integrated circuit detector such as a CMOS integrated circuit detector or a CCD integrated circuit detector. The detector according to one example can be an integrated circuit chip manufactured using integrated circuit manufacturing processes such as complementary metal oxide semiconductor (CMOS) fabrication processes.

The resolution of the biosensor array is defined as the number of pixels allocated for each reaction sight, which can be as small as 1 pixel per reaction sight, or can be greater than about 50 megapixels per reaction sight.

The detector can include a plurality of stacked layers including a sensor layer, which can be a silicon layer. The stacked layers can include a plurality of dielectric layers. In the illustrated example, each of the dielectric layers includes metallic elements (e.g. W (tungsten), Cu (copper), or Al (aluminum)) and dielectric material, e.g. Al₂O₃, Si₃N₄, SiO₂. Various metallic elements and dielectric material can be used, such as those suitable for integrated circuit manufacturing. However, in other examples, one or more of the dielectric layers can include only dielectric material, such as one or more layers of SiO₂.

In some embodiments, the field of view is from about 0.25 micron square to about 2.5 cm2. In some embodiments, the field of view can be from about 100 micron square to about 1000 mm². In some embodiments, the field of view is 5 microns by 5 microns. In some embodiments, the field of view is 100 mm by 100 mm. In some embodiments, the field of view is round. In some embodiments, the field of view is square-shaped.

### Analytes

A broad range of analytes may be detected using the disclosure herein. Exemplary analytes include small molecules, hormones, proteins, nucleic acids, carbohydrates, cells or cellular structures, virus particles or virus constituents. Presence of one analyte or type of analyte is not mutually exclusive with many other analytes, such that one may concurrently detect a broad range of analytes concurrently. In some embodiments, the analytes comprise at least some non-nucleic acid targets, or a majority of non-nucleic acid targets, or the target analytes do not comprise nucleic acids. The analytes may be similar biochemically such that they are readily enriched together. Alternately, or in combination, some of the analytes may be involved in a common signaling or other biochemical pathway.

Analytes are detected across a broad range of concentrations. In some cases, analytes are detected at a concentration of as low as or less than 1 fM, 10 fM, 100 fM, 1 pM, 10 pM, 100 pM, 1 nM, 10 nM, 100 nM or greater.

In some cases similar analytes may be independently identified, or quantified, through the disclosure herein. For example, proteins may be distinguished from one another by post-translational modification, such as phosphorylation status, glycosylation status, alkylation, lipidation, myristylation, carbonylation, glycosylation, or other post-translational modifications known in the art or otherwise identified. Similarly, nucleic acid modifications may be distinguished from unmodified variants, such as methylation, pseudouridylation, 2-O-methylation or other modification.

As an example, methods and systems herein may detect, distinguish and quantify the difference between glycosylated and total hemoglobin proteins in a blood sample (e.g., HbAlc test), phosphorylated or unphosphorylated RPS6 protein, phosphorylated or unphosphorylated cell cycle proteins such as p53, nucleosome or histone acetylation status, protein ubiquitination status, or other modifications.

Alternately, post-translational modifications or allelic variants in proteins may be identified de novo through the disclosure herein, for example by observing a shift in a target analyte binding affinity to a affinity reagent cluster relative to an expected binding affinity. That is, identification of a target analyte at a cluster that comprises aptamers that bind that target at a known binding affinity, but observing that target analyte to have a binding affinity that differs from an expected binding affinity, may indicate that the target analyte harbors a post-translational modification or an allelic variation, in the case of proteins for example, that slightly impacts without abolishing binding affinity. The shift in binding affinity can manifest itself in a shift of temperature at which fluorescence is abolished of, for example, at least, at most or about 1, 2, 3, 4, 5, 6, 7, 8, 9, 10 or more than 10 degrees Celsius, or a shift in temperature or other environmental condition or disruptive force magnitude of, for example at least, at most or about 1%, 2%, 3%, 4%, 5%, 6%, 7%, 8%, 9%, 10%, 15%, 20%, 30% or greater.

The analyte can be a small molecule, protein, carbohydrate, peptide, antigen, polymer, and the like. In some embodiments, the analyte can be a species of a Comprehensive Metabolic Panel. A typical Comprehensive Metabolic Panel (CMP) can include or exclude any of the following analytes, which can be detected using the methods described herein: Glucose, Calcium, Sodium, Potassium, Bicarbonate (an electrolyte that reflects the level of carbon dioxide (CO₂) in the sample), Chloride, Blood urea nitrogen (BUN), Creatinine, Albumin, Total protein (the sum of albumin and globulins), Alkaline phosphatase (ALP), Alanine aminotransferase (ALT), Aspartate aminotransferase (AST), and Bilirubin. Often, target analytes comprise at least some non-nucleic acid targets, or a majority of non-nucleic acid targets.

In the broadest embodiments, the disclosure herein is consistent with detection of any analyte for which an affinity reagent binding partner can be identified.

Some target analytes are subjected to repeated, iterative or ongoing detection, such as through the repeated flow of samples across the surface of an array comprising affinity reagents configured to assay for target analyte presence. An example of such a configuration is a glucose sensor, such as one that iteratively receives blood samples and assays for glucose levels in the samples.

### Numbered Embodiments

The disclosure herein is further understood in light of the following numbered embodiments. Numbered embodiments herein are understood to relate to any of the previous embodiments listed, as well as other embodiments disclosed herein.

1. A method of assaying for the presence of a binding partner in a sample, the method comprising: (a) contacting an immobilized molecule that has a binding competent state that binds a binding partner with a sample such that when the binding partner is present, the binding partner forms a complex with the immobilized molecule; (b) measuring the persistence of the binding competent state of the immobilized molecule in response to a change in its environment, (c) comparing the persistence of the binding competent state to that in the absence of said binding partner, wherein when the persistence of the binding competent state is about the same as that in the absence of the binding partner, the binding partner is absent; and wherein when the persistence of the binding competent state is different than that in the absence of the binding partner, the binding partner is present. 2. The method of any one of the previous embodiments, wherein the persistence is measured after the immobilized molecule forms the complex with the binding partner. 3. The method of any one of the previous embodiments, wherein the persistence is measured before the immobilized molecule forms the complex with the binding partner. 4. The method of any one of the previous embodiments, wherein the persistence is measured continuously while the immobilized molecule is responding to the change in its environment. S. The method of any one of the previous embodiments, wherein the change in the environment is applied in a stepwise manner. 6. The method of any one of the previous embodiments, wherein in step (b), the environment change is selected from a change of: concentration of chaotropic agents, electric field, magnetic field, ionic strength, light intensity, or shear force. 7. The method of any one of the previous embodiments, wherein the immobilized molecule in the binding competent state binds to the binding partner with a Kd of less than 10\hat{ }-6 M. 8. The method of any one of the previous embodiments, wherein the immobilized molecule is selected from an antibody, small molecule, synthetic polymer, peptide, nucleic acid, or protein. 9. The method of any one of the previous embodiments, wherein the nucleic acid is an aptamer. 10. The method of any one of the previous embodiments, wherein the aptamer is an RNA aptamer. 11. The method of any one of the previous embodiments, wherein the aptamer is a DNA aptamer. 12. The method of any one of the previous embodiments, wherein the binding partner is a small molecule, an ion, peptide, protein, synthetic polymer, antibody, cell, virus, organelles, nucleic acid, oligosaccharide, or component or fragment thereof. 13. The method of any one of the previous embodiments, wherein when the binding partner is a nucleic acid, the immobilized molecule is not a nucleic acid. 14. The method of any one of the previous embodiments, wherein the immobilized molecule is a nucleic acid, the binding partner is not a nucleic acid. 15. The method of any one of the previous embodiments, wherein the immobilized molecule has a tertiary structure. 16. The method of any one of the previous embodiments, wherein the persistence is measured by detecting a conformational change in the immobilized molecule. 17. The method of any one of the previous embodiments, wherein the binding partner is selected from an ion, small molecule, peptide, glycopeptide, carbohydrate (including oligosaccharides), protein (including glycoproteins), cell, virion, or pathogen. 18. The method of any one of the previous embodiments, wherein the immobilized molecule comprises a dye or dye pair conjugated to the immobilized molecule. 19. The method of any one of the previous embodiments, wherein the wherein the dye pair is selected from a donor-acceptor fluorophore pair. 20. The method of any one of the previous embodiments, wherein the donor-acceptor fluorophore pair is a FRET pair (Forster resonance energy transfer). 21. The method of any one of the previous embodiments, wherein the dye pair is a fluorophore-quencher pair. 22. The method of any one of the previous embodiments, wherein each of the dyes is conjugated to a separate site on the immobilized molecule. 23. The method of any one of the previous embodiments, wherein the immobilized molecule is an aptamer, and the dyes are conjugated to a modified nucleotide within the aptamer sequence. 24. The method of any one of the previous embodiments, wherein the immobilized molecule is a protein, and the dyes are conjugated to the protein by chemical linkage to one or a plurality of canonical or non-canonical proteogenic amino acids within the protein. 25. The method of any one of the previous embodiments, wherein at least one of the non-canonical proteogenic amino acids comprises a biorthogonal reactive moiety 26. The method of any one of the previous embodiments, wherein at least one of the non-canonical proteogenic amino acids provides a site for conjugation. 27. The method of any one of the previous embodiments, wherein the persistence is measured by measuring a photo-physical property (fluorescence intensity, wavelength, polarization, photoluminescence lifetime, chemiluminescence, or rotation) of one or more of the dyes of the dye pair 28. The method of any one of the previous embodiments, wherein the photophysical property is measured using an electronic sensor. 29. The method of any one of the previous embodiments, wherein the electronic sensor comprises a CMOS (complementary metal-oxide semiconductor) detector. 30. The method of any one of the previous embodiments, wherein the persistence is measured by measuring one or a plurality of physical characteristics of the immobilized molecule and the binding partner. 31. The method of any one of the previous embodiments, wherein the mass is measured using surface acoustic wave measurements, surface plasmon resonance (SPR), or bilayer interferometry (BLI). 32. The method of any one of the previous embodiments, wherein the persistence is measured by measuring the structure of the immobilized molecule. 33 The method of any one of the previous embodiments, wherein the structure of the immobilized molecule is measured using AFM (atomic force microscopy) or STM (scanning tunneling microscopy). 34. The method of any one of the previous embodiments, wherein the sample is from a bodily fluid. 35. The method of any one of the previous embodiments, wherein the bodily fluid is selected from nasal turbinate, ocular fluid, cerebral spinal fluid, urine, feces, diarrhea, bone marrow, blood, plasma, saliva, homogenized tissue, or sweat. 36. The method of any one of the previous embodiments, wherein the binding competent state of the immobilized molecule has a lower free energy state when the immobilized molecule is in a complex with the binding partner than when not in a complex with the binding partner. 37. The method of any one of the previous embodiments, wherein step (c) is performed using a computer processor. 38. A method of detecting the presence of an analyte in a sample suspected of or having said analyte, the method comprising: (a) applying an excitation light source and an electric field to an immobilized aptamer which comprises a dye pair and has one or a plurality of binding competent state(s); (b) contacting the immobilized aptamer with a sample suspected of or having said analyte such that when the analyte is present, the analyte forms a complex with the immobilized aptamer; (c) measuring the persistence of the binding competent state(s) of the immobilized aptamer in the presence of the sample upon application of an electric field; (d) comparing the persistence of the binding competent state(s) of the immobilized aptamer in the presence and the absence of the sample, wherein when the persistence of the binding competent state(s) is about the same as that in the absence of the sample, the analyte is absent; and wherein when the persistence of the binding competent state(s) is different than that in the absence of the sample, the analyte is present. 39. The method of any one of the previous embodiments, wherein comprising the steps of: (i) measuring the persistence of one or a plurality of binding competent state(s) of the aptamer in the presence of the excitation light source and applied electric field; and (ii) allowing the aptamer to return to a binding competent state(s) by removing or reducing the applied electric field. 40. A biosensor comprising: (a) an excitation light source; (b) an imaging device that is configured to convert an optical signal into an electric signal; (c) a conductive layer comprising one or a plurality of clusters of immobilized molecules each of which comprises a dye or dye pair in a medium; (d) an environmental perturbation apparatus that is configured to perturb the environment of the immobilized molecules; and (e) an optical coupling medium configured to be positioned between the one or plurality of clusters of immobilized molecules and the imaging device, wherein the excitation source optionally comprises an optical filter which defines an illumination wavelength and bandwidth, wherein the optical coupling medium is configured to transmit the optical signal from the immobilized molecule and block the background optical signal from the excitation light source, wherein the optical coupling medium optionally comprises one or a plurality of an optical element selected from: spectral filters, diffraction gratings, light reflectors, light absorbers, and total internal reflection (TIR) structures, and wherein said optical element is configured to detect an optical signal from the immobilized molecules in the presence of background illumination from the excitation light source, wherein the optical coupling medium optionally is physically connected to the imaging device and/or the conductive layer, wherein the optical coupling medium optionally comprises an optical lens or is lens-less, wherein the conductive layer is configured to transfer charges from the environmental perturbation apparatus to perturb the environment of the one or a plurality of clusters of immobilized molecules, wherein the conductive layer optionally is configured to allow light coupling from the dye or dye pair to the optical coupling medium, wherein the environmental perturbation apparatus optionally comprises a voltage sweep source which applies a time-varying voltage signal across the medium in which the one or a plurality of clusters of immobilized molecules is present, wherein the imaging device is configured to convert a received optical signal into an electrical signal, wherein the strength of the electrical signal is directly dependent on the strength of the received optical signal after pathing through the optical coupling medium, wherein the imaging device is a CMOS, CCD, or photodiode. 41. The biosensor of any one of the previous embodiments, wherein the conductive layer is optically transparent. 42. The biosensor of any one of the previous embodiments, wherein the operable coupling between the optical filter and the transparent conductive layer is a physical connection. 43. The biosensor of any one of the previous embodiments, wherein the dye pair is covalently linked to the nucleic acid sequence. 44. The biosensor of any one of the previous embodiments, wherein the field of view which is defined by the walls is essentially optically transparent to the wavelength(s) emitted by the emission light source. 45. The biosensor of any one of the previous embodiments, wherein the solid-state imager is a CMOS imager. 46. The biosensor of any one of the previous embodiments, wherein the solid-state imager is configured to detect the emission signal from the dye pair when the dye pair is subject to a light source from the excitation light source. 47. The biosensor of any one of the previous embodiments, wherein the excitation source is configured to illuminate the cluster of immobilized molecules. 48. The biosensor of any one of the previous embodiments, wherein the excitation source is configured to selectively illuminate the cluster of immobilized molecules. 49. The biosensor of any one of the previous embodiments, wherein comprising a data communication channel which is configured to transfer the electric signal data versus the voltage sweep profile to a computer processing unit which is programmed to compare the electrical signal originating from the bound and unbound aptamer clusters designed for a given target molecule. 50. An array comprising a plurality of biosensors of any one of the previous embodiments. 51. A method of assaying for an analyte in a sample, comprising: contacting the sample to an affinity reagent having a first configuration, wherein the affinity reagent first configuration is sensitive to presence of the analyte; and assaying the configuration of the affinity regent. 52. The method of any one of the previous embodiments, wherein the affinity reagent comprises an oligonucleotide. 53. The method of any one of the previous embodiments, wherein the oligonucleotide comprises an aptamer. 54. The method of any one of the previous embodiments, wherein the affinity reagent comprises a protein. 55. The method of any one of the previous embodiments, wherein protein comprises an antibody. 56. The method of any one of the previous embodiments, wherein the aptamer comprises DNA. 57. The method of any one of the previous embodiments, wherein the aptamer comprises RNA. 58. The method of any one of the previous embodiments, wherein the aptamer is tethered to a surface. 59. The method of any one of the previous embodiments, wherein the aptamer is in solution. 60. The method of any one of the previous embodiments, wherein the affinity reagent comprises a first binding moiety that binds the analyte at a first region and a second binding moiety that binds that analyte at a second region. 61. The method of any one of the previous embodiments, wherein the first binding moiety and the second binding moiety share a common phosphodiester bond. 62. The method of any one of the previous embodiments, wherein the sample is in solution. 63. The method of any one of the previous embodiments, wherein the sample is an aqueous sample. 64. The method of any one of the previous embodiments, wherein the sample is a raw sample. 65. The method of any one of the previous embodiments, wherein the sample is buffered. 66. The method of any one of the previous embodiments, wherein the assaying comprises heating the affinity reagent. 67. The method of any one of the previous embodiments, wherein the assaying comprises subjecting the affinity reagent to an electric field. 68. The method of any one of the previous embodiments, wherein the assaying comprises subjecting the affinity reagent to a magnetic field. 69. The method of any one of the previous embodiments, wherein the assaying comprises subjecting the affinity reagent to sonication. 70. The method of any one of the previous embodiments, wherein the assaying comprises subjecting the affinity reagent to acoustic waves. 71. The method of any one of the previous embodiments, wherein the assaying comprises measuring fluorescence of the affinity reagent. 72. The method of any one of the previous embodiments, wherein the assaying comprises changing a condition and measuring fluorescence during the changing of the condition. 73. The method of any one of the previous embodiments, wherein the assaying comprises changing a condition and measuring fluorescence subsequent to the changing of the condition. 74. The method of any one of the previous embodiments, wherein the aptamer comprises a fluorophore. 75 The method of any one of the previous embodiments, wherein the aptamer comprises a quencher. 76. The method of any one of the previous embodiments, wherein the aptamer comprises a fluorophore acceptor pair. 77. The method of any one of the previous embodiments, wherein a change in aptamer configuration comprises binding to the analyte. 78. The method of any one of the previous embodiments, wherein a change in aptamer configuration comprises stabilizing the aptamer configuration. 79. The method of any one of the previous embodiments, wherein a change in aptamer configuration indicates presence of the analyte in the sample. 80. The method of any one of the previous embodiments, wherein the change in aptamer configuration results in an increased aptamer stability. 81. The method of any one of the previous embodiments, wherein the change in aptamer configuration results in a decreased fluorescence. 82. The method of any one of the previous embodiments, wherein the change in aptamer configuration results in change in a threshold at which the changing of the condition impacts aptamer fluorescence. 83. The method of any one of the previous embodiments, wherein the assaying is completed in no more than minutes. 84. The method of any one of the previous embodiments, wherein the assaying is completed in no more than 30 seconds. 85. The method of any one of the previous embodiments, wherein the assaying is sensitive to an analyte at a concentration of at least 1 fM. 86. The method of any one of the previous embodiments, wherein the sample comprises blood. 87. The method of any one of the previous embodiments, wherein the sample comprises a body fluid. 88. The method of any one of the previous embodiments, wherein the sample comprises a blood droplet. 89. The method of any one of the previous embodiments, wherein the sample comprises at least 20 μL. 90. A surface comprising: a plurality of aptamer clusters, wherein a first cluster of the plurality of clusters comprises a first aptamer having a first configuration, wherein the aptamer first configuration is sensitive to presence of a first analyte; and wherein a second cluster of the plurality of clusters comprises a second aptamer having a second configuration, wherein the aptamer second configuration is sensitive to presence of a second analyte. 91. The surface of any one of the previous embodiments, wherein a third cluster of the plurality of clusters comprises an aptamer having a first configuration, wherein the aptamer first configuration is sensitive to presence of a first analyte. 92. The surface of any one of the previous embodiments, wherein a third cluster of the plurality of clusters comprises a chimeric aptamer comprising at least a binding moiety of the first aptamer and at least a binding moiety of the second aptamer. 93. The surface of any one of the previous embodiments, wherein at least some of the plurality of clusters are homogenous as to aptamer composition. 94. The surface of any one of the previous embodiments, wherein at least some of the plurality of clusters are heterogeneous as to aptamer composition. 95. The surface of any one of the previous embodiments, wherein at least one of the plurality of clusters consists of the first aptamer. 96. The surface of any one of the previous embodiments, wherein at least one of the plurality of clusters consists of the second aptamer. 97. The surface of any one of the previous embodiments, wherein at least some of the plurality of clusters comprise a single aptamer population per cluster. 98. The surface of any one of the previous embodiments, wherein an aptamer of the aptamer clusters comprises a fluorophore. 99. The surface of any one of the previous embodiments, wherein the aptamer of the aptamer clusters comprises a quencher. 100. The surface of any one of the previous embodiments, wherein the aptamer of the aptamer clusters comprises a fluorophore acceptor pair. 101. The surface of any one of the previous embodiments, wherein individual aptamers of a set of clusters of the plurality of clusters bind to a set of analytes implicated in a common biological process. 102. The surface of any one of the previous embodiments, wherein the process is a signaling pathway. 103. The surface of any one of the previous embodiments, wherein the process is a cancer pathway. 104. The surface of any one of the previous embodiments, wherein the process is a cancer progression. 105. The surface of any one of the previous embodiments, wherein binding the first analyte to the surface comprises delivering the analyte in an aqueous solution. 106. The surface of any one of the previous embodiments, wherein binding the first analyte to the surface does not require processing the analyte from a sample. 107. The surface of any one of the previous embodiments, wherein the plurality of aptamer clusters comprises at least 100 clusters. 108. The surface of any one of the previous embodiments, wherein the plurality of aptamer clusters comprises at least 200 clusters. 109. The surface of any one of the previous embodiments, wherein the plurality of aptamer clusters comprises at least 500 clusters. 110. The surface of any one of the previous embodiments, wherein the plurality of aptamer clusters comprises at least 1000 clusters. 111. The surface of any one of the previous embodiments, wherein the plurality of aptamer clusters comprises at least 2000 clusters. 112. The surface of any one of the previous embodiments, wherein the plurality of aptamer clusters comprises at least 5000 clusters. 113. The surface of any one of the previous embodiments, wherein the plurality of aptamer clusters comprises at least 10000 clusters. 114. The surface of any one of the previous embodiments, wherein the plurality of aptamer clusters each exhibit a diameter of about 10 microns. 115. The surface of any one of the previous embodiments, wherein the plurality of aptamer clusters each exhibit a diameter of about 30 microns. 116. The surface of any one of the previous embodiments, wherein the plurality of aptamer clusters each exhibit a diameter of about 500 microns. 117. The surface of any one of the previous embodiments, wherein the plurality of aptamer clusters exhibit a cluster pitch of 40 μm. 118. The surface of any one of the previous embodiments, wherein the plurality of aptamer clusters each exhibit an aptamer density of about 10e14 aptamer molecules per cm2. 119. The surface of any one of the previous embodiments, wherein the plurality of aptamer clusters each exhibit an aptamer density of about 10e11 aptamer molecules per cm2. 120. The surface of any one of the previous embodiments, wherein the plurality of aptamer clusters each exhibit an analyte bound conformational change at about the same temperature. 121. A system for analyte detection, comprising: a surface comprising a plurality of aptamer clusters; a surface condition modulator; and an imaging apparatus. 122. The system of any one of the previous embodiments, wherein the system does not comprise moving parts. 123. The system of any one of the previous embodiments, wherein the system does not comprise a microfluidics pump. 124. The system of any one of the previous embodiments, wherein the system does not comprise fluid piping. 125. The system of any one of the previous embodiments, wherein the surface is an interior of a flowcell. 126. The system of any one of the previous embodiments, wherein the aptamer clusters are present at a cluster pitch of about 40 μm. 127. The system of any one of the previous embodiments, wherein the aptamer clusters comprise aptamers of a common cluster that bind a common target. 128. The system of any one of the previous embodiments, wherein the aptamer clusters comprise aptamers that are fluorophore labeled. 129. The system of any one of the previous embodiments, wherein the aptamer clusters are quencher labeled. 130. The system of any one of the previous embodiments, wherein the aptamer clusters are FRET pair labeled. 131. The system of any one of the previous embodiments, wherein the condition modulator regulates temperature. 132. The system of any one of the previous embodiments, wherein the condition modulator regulates ion concentration. 133. The system of any one of the previous embodiments, wherein the condition modulator regulates a buffer condition. 134. The system of any one of the previous embodiments, wherein the condition modulator regulates current. 135. The system of any one of the previous embodiments, wherein the condition modulator regulates voltage. 136. The system of any one of the previous embodiments, wherein the condition modulator is a thermal block. 137. The system of any one of the previous embodiments, wherein the imaging apparatus is a digital camera. 138. The system of any one of the previous embodiments, wherein the imaging apparatus is a digital phone. 139. The system of any one of the previous embodiments, wherein the imaging apparatus is fixed to the surface. 140. The system of any one of the previous embodiments, wherein the plurality of aptamer clusters comprises at least 1,000 clusters having distinct aptamers. 141. The system of any one of the previous embodiments, wherein the plurality of aptamer clusters comprises aptamers targeting at least 1,000 distinct target analytes. 142. The system of any one of the previous embodiments, wherein the plurality of aptamer clusters comprises aptamers targeting at least 10 distinct target analytes. 143. The system of any one of the previous embodiments, wherein the plurality of aptamer clusters comprises aptamers targeting a single target analyte 144. The system of any one of the previous embodiments, wherein an analyte is detected in no more than 5 minutes. 145. The system of any one of the previous embodiments, wherein an analyte is detected in no more than 30 seconds. 146. The system of any one of the previous embodiments, wherein an analyte is detected at a concentration of at least 1 fM. 147. The system of any one of the previous embodiments, wherein the system accommodates a sample of at least 20 μL. 148. The system of any one of the previous embodiments, wherein the aptamer clusters exhibit an analyte bound conformational change at about the same temperature. 149. A method of assaying for an analyte in a sample, comprising: contacting the sample to a surface comprising a plurality of aptamer populations, changing a condition at the surface, and assaying for a change in at least one aptamer configuration. 150. The method of any one of the previous embodiments, wherein the contacting, changing a condition and assaying are completed in no more than 5 minutes. 151. The method of any one of the previous embodiments, wherein the contacting, changing a condition and assaying are completed in no more than 30 seconds. 152. The method of any one of the previous embodiments, wherein the assaying distinguishes presence of the analyte at a concentration of as low as 1 fM. 153. The method of any one of the previous embodiments, wherein the assaying may detect as many as 1,000 analytes on a single surface. 154. The method of any one of the previous embodiments, wherein the analyte comprises a protein. 155. The method of any one of the previous embodiments, wherein the method distinguishes the protein according to a post-translational state of the protein. 156. The method of any one of the previous embodiments, wherein the analyte comprises a phosphorylation state of the protein. 157. The method of any one of the previous embodiments, wherein the analyte comprises a small molecule. 158. The method of any one of the previous embodiments, wherein the analyte comprises a metabolite. 159. The method of any one of the previous embodiments, wherein the analyte comprises a carbohydrate. 160. The method of any one of the previous embodiments, wherein the analyte comprises a nucleic acid 161. The method of any one of the previous embodiments, wherein the analyte comprises a lipid. 162. The method of any one of the previous embodiments, wherein the analyte comprises an epitope. 163. The method of any one of the previous embodiments, wherein the analyte comprises a cell. 164. The method of any one of the previous embodiments, wherein the analyte comprises a virus. 165. A method of assaying for an analyte in a sample, comprising: contacting the sample to a surface comprising a plurality of aptamer populations, assaying for aptamer population first fluorescence, changing a condition at the surface, and assaying for aptamer population second fluorescence. 166. The method of any one of the previous embodiments, wherein changing a condition comprises a gradual changing. 167. The method of any one of the previous embodiments, wherein assaying for aptamer population second fluorescence comprises gradual assaying. 168. The method of any one of the previous embodiments, wherein changing a condition comprises a continuous changing. 169. The method of any one of the previous embodiments, wherein changing a condition comprises a discrete changing. 170. The method of any one of the previous embodiments, wherein the surface comprises wells, and wherein the aptamer populations are segregated into wells. 171. The method of any one of the previous embodiments, wherein the surface comprises wells, and wherein the aptamer populations are immobilized on beads, and wherein the beads are localized into the wells. 172. The method of any one of the previous embodiments, wherein the wells accommodate no more than one bead per well. 173. The method of any one of the previous embodiments, comprising sequencing or decoding a tag associated with a bead in a well. 174. The method of any one of the previous embodiments, comprising sequencing or decoding an aptamer associated with a bead in a well. 175. A method of distinguishing among analytes in a sample, comprising binding an unknown analyte to an aptamer that binds a first analyte and a second analyte, changing a condition at the analyte and concurrently measuring fluorescence at the analyte, and observing a change in fluorescence, wherein the first analyte causes a change in aptamer fluorescence at a first change in the condition, and the second analyte causes a change in aptamer fluorescence at a second change in the condition. 176. The method of any one of the previous embodiments, wherein the aptamer is tethered to a surface. 177. The method of any one of the previous embodiments, wherein the surface is covered with a liquid at the aptamer. 178. The method of any one of the previous embodiments, wherein the first analyte and the second analyte are proteins having identical polypeptide sequence but differ in a post-translational modification. 179. The method of any one of the previous embodiments, wherein the post translational modification is a phosphorylation. 180. The method of any one of the previous embodiments, wherein the condition is temperature. 181. The method of any one of the previous embodiments, wherein the aptamer comprises a fluorophore. 182. The method of any one of the previous embodiments, wherein the aptamer comprises a quencher. 183. The method of any one of the previous embodiments, wherein the aptamer comprises an acceptor pair. 184. The method of any one of the previous embodiments, wherein the aptamer is immobilized in a well. 185. The method of any one of the previous embodiments, wherein the aptamer is tethered to a bead that is localized to a well. 186. The method of any one of the previous embodiments, wherein the bead comprises an oligo tag. 187. The method of any one of the previous embodiments, comprising sequencing or decoding the oligo tag. 188. The method of any one of the previous embodiments, comprising sequencing or decoding the aptamer tethered to the bead in the well. 189. A method of quantifying an analyte in a sample, comprising contacting the sample to a surface comprising a plurality of affinity clusters, wherein the aptamer clusters bind the analyte in a condition-dependent manner, and assaying for first fluorescence at a first condition value. 190. The method of any one of the previous embodiments, wherein the affinity clusters comprise aptamers. 191. The method of any one of the previous embodiments, wherein the condition is temperature. 192. The method of any one of the previous embodiments, wherein the condition is voltage. 193. The method of any one of the previous embodiments, wherein the condition is current. 194. The method of any one of the previous embodiments, wherein the condition is ionic concentration. 195. The method of any one of the previous embodiments, comprising diluting the sample prior to contacting. 196. The method of 12 any one of the previous embodiments, comprising changing the condition, and assaying for fluorescence at a second condition value. 197. The method of any one of the previous embodiments, wherein the number of aptamer clusters exhibiting aptamer-bound fluorescence effects quantifying the analyte in the sample. 198. The method of any one of the previous embodiments, wherein the aptamer comprises a fluorophore. 199. The method of any one of the previous embodiments, wherein the aptamer comprises a quencher. 200. The method of any one of the previous embodiments, wherein the aptamer comprises an acceptor pair. 201. The method of any one of the previous embodiments, wherein at least a subset of the plurality of aptamer clusters bind a common analyte. 202. The method of any one of the previous embodiments, wherein the at least a subset of the plurality of aptamer clusters comprise distinct populations of nonidentical aptamers. 203. The method of any one of the previous embodiments, wherein the nonidentical aptamers exhibit nonidentical analyte affinities. 204. The method of any one of the previous embodiments, wherein the at least a subset of the plurality of aptamer clusters comprise aptamers having distinct surface tethering moieties. 205. The method of any one of the previous embodiments, wherein the aptamers having distinct surface tethering moieties exhibit nonidentical analyte affinities. 206. The method of any one of the previous embodiments, wherein the aptamer clusters are immobilized to the surface. 207. The method of any one of the previous embodiments, wherein the aptamers are localized to wells on the surface 208 The method of any one of the previous embodiments, wherein the aptamers are bound to beads, and the beads are localized to wells on the surface. 209. The method of any one of the previous embodiments, wherein the beads comprise bead-identifying oligo tags. 210. The method of any one of the previous embodiments, wherein sequencing or decoding the oligo tag. 211. The method of any one of the previous embodiments, wherein sequencing or decoding the aptamers tethered to the beads in the well. 212. A system comprising i) a surface comprising a plurality of aptamer clusters, ii) a heating unit, iii) an imager, wherein the system detects at least 100 distinct non-nucleic acid target analytes at a concentration of as low as 100 fM in no more than 10 minutes. 213. The system of any one of the previous embodiments, wherein the system detects at least 1000 distinct non-nucleic acid target analytes. 214. The system of any one of the previous embodiments, wherein the system detects at least 10,000 distinct non-nucleic acid target analytes. 215. The system of any one of the previous embodiments, wherein the system detects the analytes at a concentration of as low as 10 fM. 216. The system of any one of the previous embodiments, wherein the system detects the analytes at a concentration of as low as 1 fM. 217. The system of any one of the previous embodiments, wherein the system detects the analytes in no more than 7 minutes. 218. The system of any one of the previous embodiments, wherein the system detects the analytes in no more than 5 minutes. 219. The system of any one of the previous embodiments, wherein the system detects the analytes in no more than 3 minutes. 220. The system of any one of the previous embodiments, wherein the system detects the analytes in no more than 1 minute. 221. The system of any one of the previous embodiments, wherein the system detects the analytes in no more than 30 second.

Accordingly, disclosed herein are compositions, devices and methods relating to use of the affinity reagents and detection of the analytes using, for example, the arrays and detection approaches disclosed herein alone or in combination with technologies otherwise known in the art.

Turning to the figures, one sees the following.

At FIG. 1, one sees a representative method of the invention wherein heat-induced unfolding of an aptamer labeled with a donor (D)-quencher (Q) pair is depicted. Gray circle represents an analyte molecule bound to an aptamer. The fluorescence as a function of application of heat (or an electric field) changes in response to the presence of the analyte molecule.

At FIG. 2, one sees immobilized aptamer clusters (each cluster is indicated with a gray circle) arrayed on an imaging sensor comprising an array of photodiodes (e.g., pixels depicted as small squares).

At FIG. 3, one sees the cross-section of a biosensor with multiple photodiode elements underneath the aptamer clusters. The transparent conductive layer is a heating element.

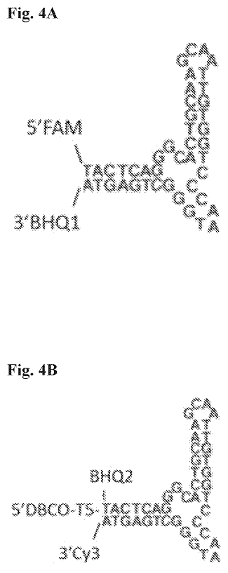

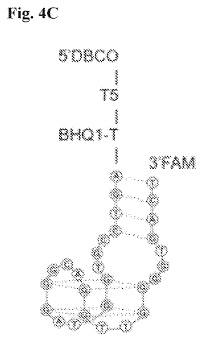

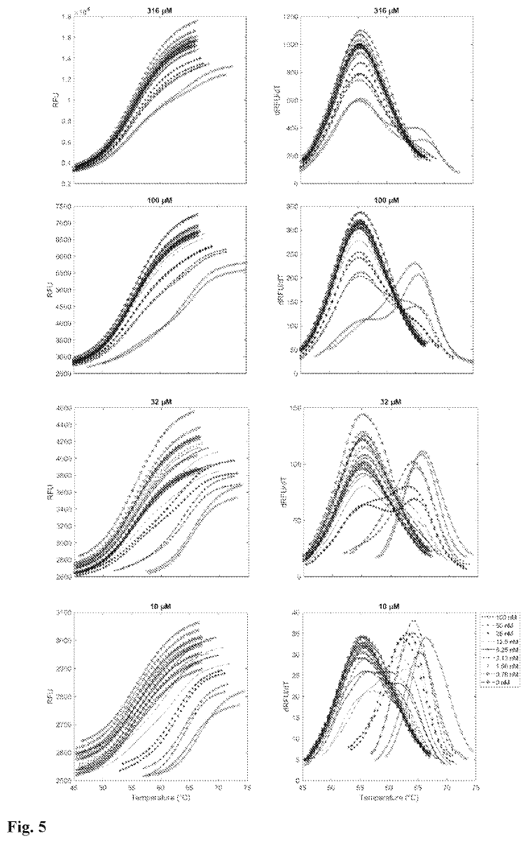

At FIG. 4A-C, one sees aptamer sequence and structure. (FIG. 4A) Sequence and secondary structure of T41 aptamer for PDGF-BB used for solution binding studies (example 1) with two terminal modifications: fluoresceine dye (FAM) at the 5′ end and Black Hole Quencher 1 (Q) at 3′ end. (FIG. 4B) Sequence and secondary structure of T41 aptamer used for binding 6 studies with the immobilized aptamer (example 3) with the following modifications: DBCO-TEG (DBCO) attached via five-thymidine linker (T5) to 5′ end; thymine-linked Black Hole Quencher 2 (BHQ2); and cyanine 3 dye (Cy3) at 3′ end. (FIG. 4C) Sequence and secondary structure of HD22 aptamer for thrombin used for solution binding studies (example 2) with the following modifications: DBCO-TEG (DBCO) attached via five-thymidine linker (T5) to 5′ end; thymine-linked Black Hole Quencher 1 (BHQ1); and fluoresceine dye (FAM) at 3′ end.

At FIG. 5, one sees solution melting curves (RFU=relative fluorescent unit, left panels) and the first derivative (dRFU/dT, right panels) of thermal titration experiments with different concentrations of T41 aptamer depicted in FIG. 4A (from top to bottom: 316 nM, 100 nM, nM, and 10 nM) with varying concentrations of PDGF-BB (from 0 to 100 nM, see legend at 16 the right bottom panel indicating 100 nM, 50 nM, 25 nM, 12.5 nM, 6.25 nM, 3.13 nM, 1.56 nM, 0.78 nM and 0 nM). For the left panels, RFU values vary for each experiment, ranging from 2,000 to 18,000 at the top panel, 2,500 to 7,500 in intervals of 500 in the second panel, 2,600 to 4,600 in intervals of 200 in the third panel, and from 2500 to 3100 in 100 unit intervals the bottom panel. At right, y-axis units range from 0 to 1200 in intervals of 200 at the top right, 0 to 350 in intervals of 50 in the second panel, 0 to 150 in intervals of 50 in the third panel, and 0 to 40 in intervals of 5 in the lower right panel. For all left and right panels, the x-axis is temperature in Celsius, and ranges from 45 to 75 in intervals of 5.

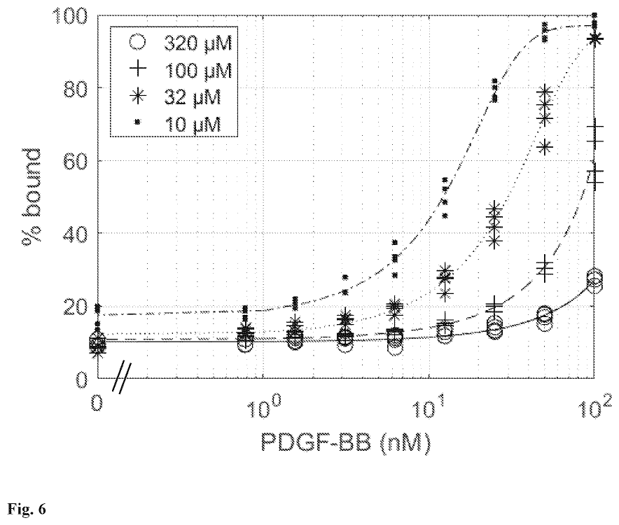

At FIG. 6, one sees dose response of PDGF-BB at different concentration of 41T aptamer (depicted in FIG. 4A) in solution. The y-axis is percentage of aptamer molecules bound to PDGF-BB (% bound), ranging from 0 to 100% in intervals of 20, while the x-axis is the concentration of PDGF-BB in nM and ranges from 0 to 100 on a logarithmic scale of 0, 1, 10, and 100. See example 1 for detailed experimental description.

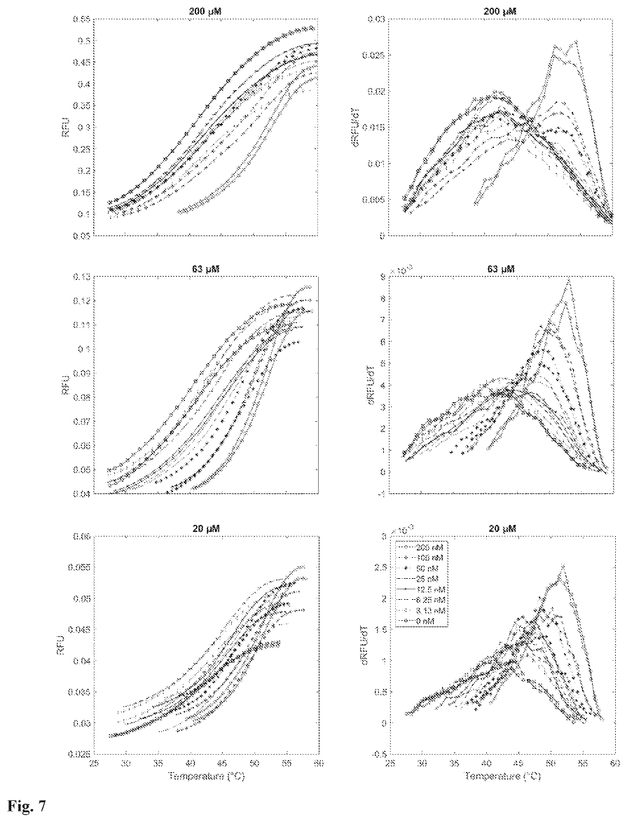

At FIG. 7, one sees solution melting curves (RFU=relative fluorescent unit, left panels) and the first derivative (dRFU/dT, right panels) of thermal titration experiments with different concentrations of HD22 aptamer depicted in FIG. 4C (from top to bottom: 200 nM, 63 nM, and 20 nM) with varying concentrations of thrombin (from 0 to 200 nM, see legend at the right bottom panel indicating 100 nM, 50 nM, 25 nM, 12.5 nM, 6.25 nM, 3.13 nM, 1.56 nM, 0.78 nM and 0 nM). For each figure, the x-axis represents temperature in Celsius, and ranges from 25 to 60 in units of 5. For the left file of figures, the y-axis represents RFU, ranging from 0.05 to 0.55 in units of 0.05 at upper left, 0.04 to 0.13 in units of 0.01 in the middle left figure, and from 0.025 to 0.06 in intervals of 0.005 in the lower left figure. For the right set of images, the y-axis is dRFU/dT, and ranges from 0 to 0.03 in units of 0.005 at upper right, −1 to 9 in intervals of 1 in the middle image, and −0.5 to 3.0 in intervals of 0.5 in the lower right image.

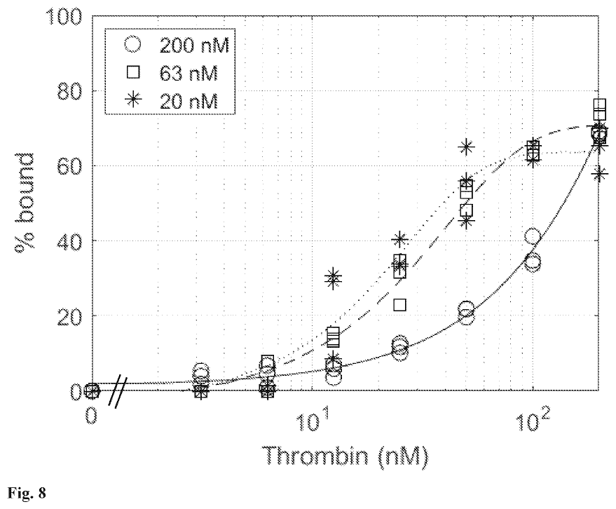

At FIG. 8, one sees dose response of thrombin at different concentration of HD22 aptamer (depicted in FIG. 4C) in solution. The y-axis is percentage of aptamer molecules bound to thrombin (% bound), ranging from 0 to 100% in intervals of 20, while the x-axis is the concentration of thrombin in nM and ranges from 0 to 100 on a logarithmic scale of 0, 10, and 100. See example 2 for detailed experimental description.

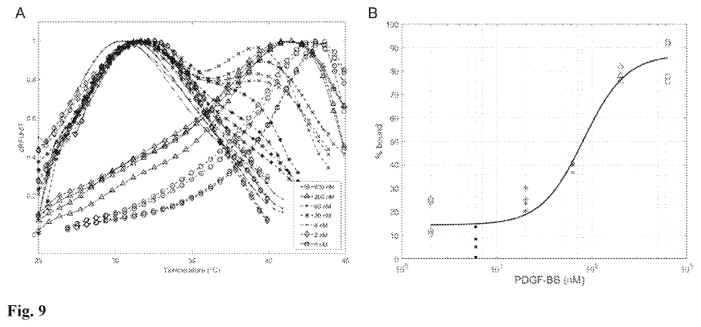

At FIG. 9, one sees normalized first derivative of the melting curves (panel A) and the calculated dose response (panel B) of the immobilized 41T aptamer depicted in FIG. 4B and varying concentrations of PDGF-BB (from 0 to 630 nM, see legend at the right bottom corner of panel A indicating concentrations of 630 nM, 200 nM, 63 nM, 20 nM, 6 nM, 2 nM and 0 nM). Units for panel A are dRFU/dT on the y-axis, in intervals of 0.2 ranging from 0 to 1, with the x-axis representing temperature in Celsius ranging from 25 to 45 in intervals of 5. At right one sees percentage of aptamer molecules bound to PDGF-BB (percent bound) on the y-axis, ranging from 0 to 100 in intervals of 10, with the x-axis is the concentration of PDGF-BB in nM and ranges exponentially from 1, 10, 100, to 1000.

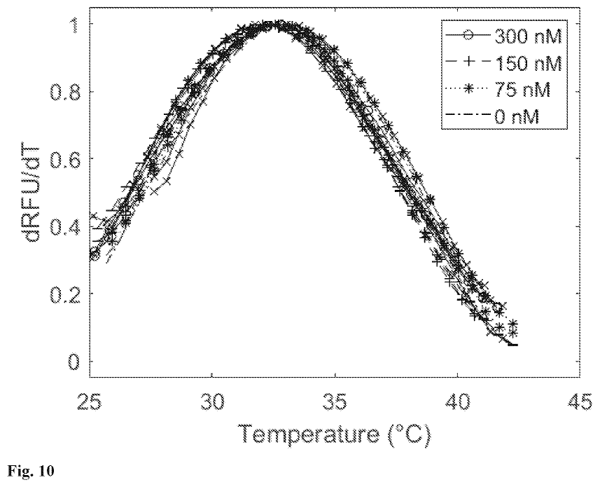

At FIG. 10, one sees normalized first derivative of the melting curves of the immobilized 41T aptamer depicted in FIG. 4B with varying concentrations of PDGF-AA (from 0 to 300 nM, see legend at the right top corner). PDGF-AA is a homolog and is structurally similar to PDGF-BB. Units dRFU/dT on the y-axis, in intervals of 0.2 ranging from 0 to 1, with the x-axis representing temperature in Celsius ranging from 25 to 45 in intervals of 5.

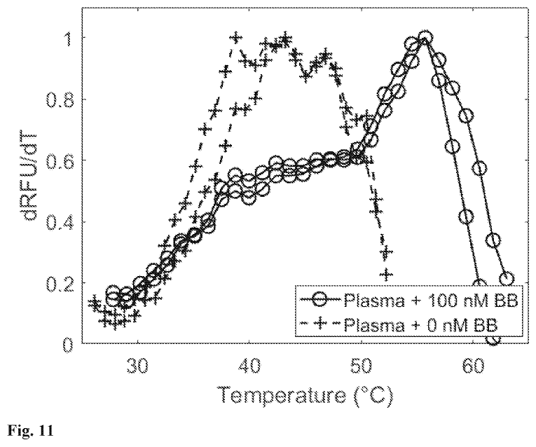

At FIG. 11, one sees normalized first derivative of melting curves (dRFU/dT) collected in human plasma in the absence (plus sign markers, dashed line) and the presence of 100 nM PDGF-BB (circles, straight line). Y-axis units are dRFU/dT, in intervals of 0.2 ranging from 0 to 1, with the x-axis representing temperature in Celsius ranging from 25 to 65 in intervals of 10.

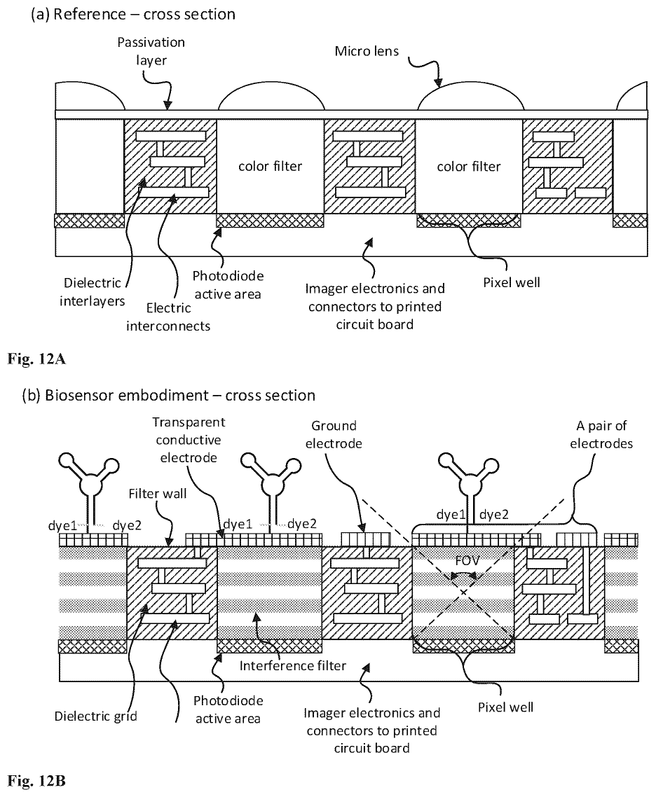

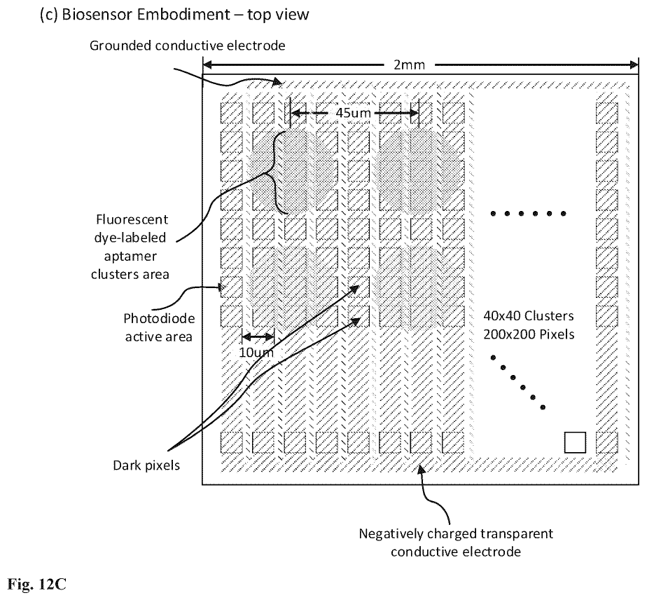

At FIG. 12A-C, one sees (FIG. 12A) Cross-section of a reference front-side illumination CMOS imaging sensor array, (FIG. 12B) cross-section of a CMOS biosensor as a representative embodiment of the invention, (FIG. 12C) top view of a CMOS biosensor as a representative embodiment of the invention.

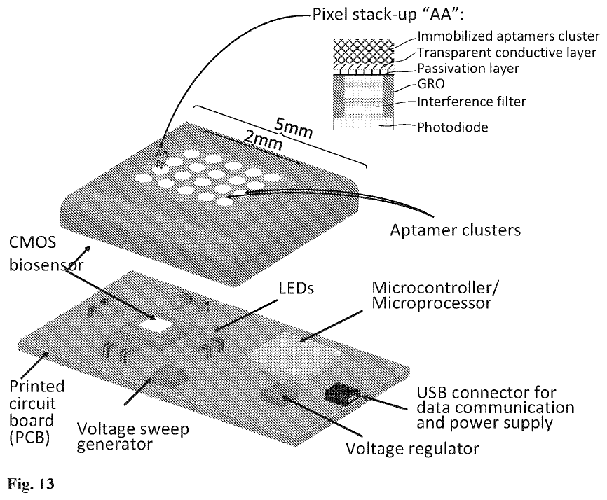

At FIG. 13, one sees a representative embodiment of a system comprising a biosensor mounted on a printed circuit board of a diagnostic device this disclosure (not to scale).

At FIG. 14, one sees a dimeric aptamer complex comprising two distinct target binding moieties that bind to a target at two distinct regions is shown. The dimeric aptamer comprises a single phosphodiester backbone (thin line) and is tethered to a surface at the region of the phosphodiester backbone that links the two portions of the dimeric aptamer by a tethering 12 moiety (thick oval). A cluster of this dimeric aptamer population comprises two distinct binding moieties but is nonetheless a uniform cluster, and exhibits a Kd that is greater than either of the binding moieties on its own.

### EXAMPLES

### Example 1-Persistence Measurement of 41T Aptamers with Varying Concentration of Binding Partners in Solution

In one representative experiment, aptamer comprising a competent binding state and a dye pair (fluoresceine and black hole quencher) was measured for their persistence in the changed environment of temperature as described herein. As a representative embodiment of a biomolecule having a competent state, aptamer binding the 41T TACTCAGGGCACTGCAAGCAATTGTGGTCCCAATGGGCTGAGTA (SEQ ID NO. 1) (Green et all, Biochemistry, Vol. 35, No. 45, 1996), which selectively binds PDGF-BB (platelet-derived growth factor-BB) was modified with fluoresceine (F) and Black hole quencher 1 (Q) at 5′ and 3′ ends, respectively (FIG. 4A) (obtained from LCG Biosearch Technologies (Petaluma, CA)), has an affinity for PDGF-BB with K_{d} of 0.13 nM. PDGF-BB, bovine serum albumine (BSA), magnesium chrolide, and phosphate buffered saline (PBS) all other buffers were ordered from Sigma-Millipore. 41T aptamer at a concentration of either 316 nM, or 100 nM, or 31.6 nM, or 10 nM were mixed with variable concentrations PDGF-BB (from 100 nM to 0.78 nM with 3.16× dilution factor) in 1× PBS containing 1 mM MgCl₂, and 0.1 mg/ml BSA on white 384 well PCR plate (BioRad) and subjected to the following thermal protocol on BioRad CFX384 qPCR instrument: lid at 95° C., melting curve step from 30° C. to 80° C. with images (FAM/SYBR channel) every 0.5° C., 10 seconds per step. FIG. 5 presents melting curves (left panels) and the first derivative (right panels) of thermal titration experiments. To estimate the relative populations of free aptamers and aptamers bound to the target (% bound), the areas under the curve of the first derivative (dRFU/dT) were integrated for both unbound (left peak with Tₘ˜55° C.) and bound (right peak with Tₘ˜65° C.) peaks. % bound as a function of the target concentration is presented as a dose response in FIG. 6.

### Example 2-Persistence Measurement of HD22 Aptamers with Varying Concentration of Binding Partners in Solution

In another example, the aptamer HD22 AGTCCGTGGTAGGGCAGGTTGGGGTGAC (SEQ ID NO: 2) (Tasset et all, J. Mol. Biol., Vol. 272, No. 688, 1997), which selectively binds thrombin was modified with DBCO-TEG attached via five-thymidine linker to 5′ end; thymine-linked Black Hole Quencher 1; and fluoresceine dye (FAM) at 3′ (FIG. 4C) (obtained from ATDBio, Southhampton, UK), has an affinity for thrombin with K_{d} of 0.5 nM. Thrombin, bovine serum albumine (BSA), magnesium chrolide, and phosphate buffered saline (PBS) all other buffers were ordered from Sigma-Millipore. HD22 aptamer at a concentration of either 200 nM, or 63 nM, or 20 nM were mixed with variable concentrations thrombin (from 200 nM to 3.1 nM with 2× dilution factor) in 1× PBS containing 1 mM MgCl2, and 0.1 mg/ml BSA on white 384 well PCR plate (BioRad) and subjected to the following thermal protocol on BioRad CFX384 qPCR instrument: lid at 95° C., melting curve step from 30° C. to 80° C. with images (FAM/SYBR channel) every 0.5° C., 10 seconds per step. FIG. 7 presents melting curves (left panels) and the first derivative (right panels) of thermal titration experiments. To estimate the relative populations of free aptamers and aptamers bound to the target (% bound), the areas under the curve of the first derivative (dRFU/dT) were integrated for both unbound (left peak with Tₘ˜42° C.) and bound (right peak with Tₘ˜52° C.) peaks. % bound as a function of the target concentration is presented as a dose response in FIG. 8.

These results of the examples 1 and 2 demonstrate that the fluorophore-quencher pair can be used to monitor analyte-dependent unfolding using temperature as an environmental perturbation.

### Example 3 Surface-Bound Aptamer Persistence Measurement of 41T Aptamer in Presence and Absence of Binding Partner

In another representative experiment, surface-immobilized aptamer comprising a competent binding state and a dye pair was measured for their persistence in the changed environment of temperature as described herein. As a representative embodiment of a biomolecule having a competent binding state, the aptamer 41T TACTCAGGGCACTGCAAGCAATTGTGGTCCCAATGGGCTGAGTA (SEQ DD NO: 1) (Green et all, Biochemistry, Vol. 35, No. 45, 1996), which selectively binds PDGF-BB (platelet-derived growth factor-BB), was obtained with the following modifications: DBCO-TEG (for covalent coupling to a surface) attached via five-thymidine linker to 5′ end; thymine-linked Black Hole Quencher 2; and cyanine 3 dye at 3′ end (FIG. 4B) from ATDBio (Southampton, UK). The solid support consisted of coated ITO glass slides functionalized with azido groups (Sikemia, Grabels, France) to form a covalent bond between the DBCO-labeled aptamer and the surface. PDGF-BB and PDGF-AA were obtained from R&D Systems (Minneapolis, USA). Human serum albumine (HSA), phosphate buffered saline (PBS) and all other reagents were ordered from Sigma-Millipore. The aptamer was covalently linked to the solid support by spotting 4.5 μL of the 0.25 uM aptamer solution in 1× PBS buffer. The spots were incubated for 10 minutes to allow for surface immobilization and washed with 1× PBS buffer. Solutions with a variable amount of PDGF-BB or PDGF-AA (630 nM to 2 nM 3.16× dilution series or 300 nM to 75 nM 2× dilution series, respectively) and containing 0.75 mg/mL. HSA, 40% DMSO (v/v) were applied to the surface coated with the aptamers and incubated for 5 minutes at room temperature. The temperature was increased to 45° C. by placing the slide on a heating element. The change in fluorescence intensity of the modified aptamer was as a function of temperature was monitored by fluorescence microscope coupled to a CMOS sensor. FIGS. 9A and 10 presents normalized first derivative of the melting curves for PDGF-BB or PDGF-AA titration experiments, respectively. To estimate the relative populations of free aptamers and aptamers bound to the target (% bound), the areas under the curve of the first derivative (dRFU/dT) were integrated for both unbound (left peak with Tₘ˜32° C.) and bound (right peak with Tₘ˜42° C.) peaks. % bound as a function of PDGF-BB concentration is presented as a dose response in FIG. 8B. No binding and no dose response of PDGF-AA to the surface-bound aptamer were detected (FIG. 10), which demonstrates high specificity of 41T aptamer specificity of its target PDGF-BB. To demonstrate target binding in a complex biological mixture, citrated human plasma powder (from 1 mL of pooled blood) was obtained from Sigma-Millipore and reconstituted with 1 mL of water. PDGF-BB was spiked into the plasma to 100 nM final concentration and the melting curves were recovered as described above except for no DMSO was present in the solution applied to the immobilized aptamer. Normalized first derivative of melting curves collected in human plasma in the absence and the presence of 100 nM PDGF-BB are shown in FIG. 11. The absence and the appearance of the bound peak (Tₘ˜55° C.) in the absence and presence of PDGF-BB, respectively, demonstrates high specificity of the 41T aptamer for its target, even in a complex biological sample.

### Example 4—CMOS Chip Design and Integration

This disclosure provides for a biosensor comprising a plurality of stacked layers and an excitation light source. The plurality of stacked layers can be configured so as to be a lens-less fluorescence imager with an induced E-field. A lens-less (contact) fluorescence imaging scheme is devised to monitor the changes in fluorescence wavelength or intensity of a fluorescent dye-labeled aptamer clusters as they transition back and forth between their binding competent to binding incompetent states and to determine the voltage where the transitions are observed (Vₘ^{ub} and Vₘ^{obs}). Such an imaging scheme can be used in a configuration comprising a donor/quencher or a donor/acceptor. A fluorescence sensing detector array can be made by the methods described herein. A commercial CMOS image sensor may include a photodiode for sensing externally-incident light, and a circuit for converting the sensed light into an electrical signal and digitizing the electrical signal. Such a CMOS image sensor may include a plurality of photodiodes formed on a semiconductor substrate, a plurality of color filters formed to correspond to the photodiodes in order to pass light in specific wavelengths, and a plurality of lenses formed to correspond to the color filters (US 2010/0103288). In our application an emission filter can be fabricated depositing alternating dielectric layers such as SiO₂ and TiO₂ (e.g., interference filter, FIG. 12B) as an alternative to the color filters in commercial applications (FIG. 12A). In addition, the transparent conductive electrodes are fabricated by depositing a patterned layer of ITO over the CMOS as an alternative to the microlenses shown in the reference configuration (FIG. 12C). Using lithographic chip fabrication, a transparent conducting layer can be deposited and patterned in an interdigital layout with a ground conductive electrode in a fashion similar to that used with interdigital microstrip capacitors. Each pair of fingers of the interdigital pattern can form a pair of E-field electrodes and cover a row/column of the pixel array. The number of electrode pairs can equal the number of rows/columns of the pixel array. The overlap of the fingers with the pixel wells can define a transparent conducting patch (TCP) array which will be biased via a common voltage sweep generator.

In some embodiments, the plurality of stacked layers is configured to be parallel to the optical filter(s). In some embodiments, the sensor can comprise an electrode pair. The electrode pair can comprise a transparent conductive electrode and a ground electrode. In some embodiments, the sensor can further comprise a reference electrode. In some embodiments, the electrode pair are configured to be on the about same surface as the immobilized molecule, such that when an applied voltage is present the immobilized molecule will be subject to the resulting electric field. In such a configuration, the applied electric field is in a “X-Y” direction. The advantage of this configuration is facile construction of the sensor and accurate measurements because the sensor can be made within a single “chip”. In some embodiments, the excitation light source is configured to be adjacent to the photodiode active area.

The physical construction of the filter wall includes metal layers with the dielectric interlayers, which is typically utilized to reduce optical crosstalk between neighboring photodiodes in typical imaging applications. For the purpose of this application, the filter wall is utilized to limit the FOV of the photodiode and allow side illumination of the dyes from the excitation source without direct exposure of the photodiodes. The FOV is determined by the height h and width w of the interference filter (FIG. 12B).

As a representative embodiment, an imaging sensor has an overall area of 5 mm×5 mm and with an active photodiode array area of 2 mm×2 mm, on which aptamer clusters will be printed (FIG. 13). The imaging sensor is bond-wired and packaged, then soldered to a printed circuit board (PCB) that integrates all the complementary electronic components needed for the biosensing device, such as USB adaptor, voltage regulars, light emitting diodes (LEDs), LED drivers, voltage sweep generator, and digital controller/processor. The LEDs may be mounted around the CMOS sensor to illuminate the aptamer cluster while being outside the FOV of the pixel array. The diameter of an aptamer cluster will be ˜30 micron, while the size of a CMOS pixel is 10 micron×10 micron. Hence, each aptamer cluster will cover 3×3 “active” pixels surrounded by a perimeter of “dark” pixels that will form a grid of 10 micron-wide reference lines and eliminate the need for sophisticated CMOS pixel-cluster alignment.

Aptamers have an intrinsic negative charge. Thus, the polarity of the applied voltage at the TCP required to induce unfolding is negative. The E-field will be swept from zero to a field strength sufficient to unfold all aptamers. When the E-field force is sufficient to induce unfolding at a specific cluster of aptamers, their fluorescence will change as described above. A portion of the isotropic emission from the aptamer will transmit through the bandpass emission filter, while all undesired radiation, including the LED scattered light, is blocked. Thus, the active pixels will detect a change in fluorescence that defines an unfolding voltage (Vₘ^{ub} or Vₘ^{obs}). As discussed above, the presence of a target analyte (binding partner), the binding competent state of an aptamer is stabilized, and thus Vₘ^{obs} will be greater than Vₘ^{ub}.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognize that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1.-221. (canceled)

222. A system for analyte detection, the system comprising: a surface comprising a plurality of affinity reagent clusters arrayed thereupon, the plurality of affinity reagent clusters comprising a first affinity reagent cluster of first affinity reagents that denature at a first unbound denaturing temperature in the absence of a first analyte, and that denature at a first bound denaturing temperature that is different than the first unbound denaturing temperature upon binding to the first analyte, and a second affinity reagent cluster of second affinity reagents that denature at a second unbound denaturing temperature in the absence of a second analyte, and that denature at a second bound denaturing temperature that is different than the second unbound denaturing temperature upon bonding to the second analyte.

223. The system of claim 222, wherein the system further comprises a surface condition modulator.

224. The system of claim 223, wherein the surface condition modulator is a thermal modulator that is capable of subjecting the surface to a temporal temperature gradient spanning the first unbound denaturing temperature, the first bound denaturing temperature, the second unbound denaturing temperature, and the second bound denaturing temperature.

225. The system of claim 222, wherein the system further comprises a camera to image the surface.

226. The system of claim 222, wherein the system further comprise a lensless CMOS sensor.

227. The system of claim 225, wherein the camera is configured to image the surface at distinct time points for an assay.

228. The system of claim 225, wherein the camera is configured to image the surface continuously for a portion of an assay.

229. The system of claim 222, wherein the surface is an interior of a flowcell.

230. The system of claim 222, wherein the affinity reagent clusters are present on beads, and the affinity reagent clusters are randomly arrayed upon the surface through the beads being deposited on the surface.

231. The system of claim 222, wherein the affinity reagent clusters comprise aptamer clusters that are fluorophore labeled.

232. The system of claim 231, wherein the aptamer clusters are quencher labeled.

233. The system of claim 231, wherein the aptamer clusters are FRET pair labeled.

234. The system of claim 222, wherein the system provides simultaneous readout for each target.

235. The system of claim 222, wherein the system provides quantitative readout for each target.

236. The system of claim 222, wherein the plurality of affinity reagent clusters comprises aptamers targeting at least 10 distinct target analytes.

237. The system of claim 236, wherein the aptamers targeting at least 10 distinct target analytes comprises aptamers that target at least two target analytes in a common signaling pathway.

238. The system of claim 236, wherein the aptamers targeting at least 10 distinct target analytes comprises aptamers that target at least two target analytes implicated in a common physiological status.

239. The system of claim 222, wherein an analyte is detected in no more than 10 minutes, enabling near real-time diagnostic decision-making at the point of care.

240. The system of claim 222, wherein an analyte is detected at a concentration of as low as 1 fM.

241. The system of claim 222, wherein denaturation is monitored via a fluorescence signal modulated by temperature-induced affinity reagent conformational changes.
